(19) United States
(12) Patent Application Publication
(10) Pub. No.: US 2024/0181083 A1
(43) Pub. Date: Jun. 6, 2024

(54) AAVRH74 VECTORS FOR GENE THERAPY OF MUSCULAR DYSTROPHIES

(71) Applicant: University of Florida Research Foundation, Incorporated, Gainesville, FL (US)

(72) Inventors: Arun Srivastava, Gainesville, FL (US); Keyun Qing, Gainesville, FL (US); Barry John Byrne, Gainesville, FL (US)

(73) Assignee: University of Florida Research Foundation, Incorporated, Gainesville, FL (US)

(21) Appl. No.: 18/287,711

(22) PCT Filed: Apr. 22, 2022
(86) PCT No.: PCT/US2022/025916

§ 371 Date: Oct. 20, 2023

Related U.S. Application Data
(60) Provisional application No. 63/327,410, filed on Apr. 5, 2022.
(60) Provisional application No. 63/179,097, filed on Apr. 23, 2021.

(51) Int. Cl. A61K 48/00; C07K 14/005; C12N 15/86

(52) U.S. Cl. CPC A61K 48/0058; A61K 48/0075; C07K 14/005; C12N 15/86; C12N 2750/14122; C12N 2750/14143; C12N 2750/14145

(57) ABSTRACT

Provided herein are modified AAV capsid proteins, particles, nucleic acid vectors, and compositions thereof, as well as methods of their use.

### RELATED APPLICATIONS

This application claims the benefit under 35 U.S.C. § 119(e) of U.S. Provisional Patent Application No. 63/179,097, filed Apr. 23, 2021 and U.S. Provisional Patent Application No. 63/327,410, filed Apr. 5, 2022, the entire contents of each of which are hereby incorporated by reference.

### REFERENCE TO SEQUENCE LISTING SUBMITTED AS A TEXT FILE VIA EFS-WEB

The instant application contains a sequence listing which has been submitted in ASCII format via EFS-Web and is hereby incorporated by reference in its entirety. Said ASCII copy, created on Apr. 22, 2022, is named U120270077WO00-SEQ-COB and is 119,114 bytes in size.

### BACKGROUND

Gene therapy has the potential to treat subject suffering from or are at risk of suffering from genetic disease. Improved AAV vectors for carrying genetic payload would be beneficial to the development of gene therapies, e.g., for certain diseases that affect muscle tissue and/or function. Muscle diseases, such as muscular dystrophies, can result from numerous conditions including, for example, congenital or acquired somatic mutations, injury, and exposure to hazardous compounds. In some cases, muscle diseases result in life-threatening complications or lead to serious symptoms and/or death. Although numerous factors have been implicated in regulating muscle diseases, including muscular dystrophies, effective treatments remain limited.

### SUMMARY

The present disclosure is based at least in part on the realization that certain amino acid substitutions in one or more capsid proteins of a recombinant AAVrh74 particle, and/or modification of an AAV nucleic acid vector encapsidated by an AAVrh74 capsid results in improved properties (e.g., transduction of particular types of cells) relative to a wild-type AAVrh74 particle or unmodified AAV nucleic acid vector encapsidated by an AAVrh74 capsid. Modifications of capsid proteins (e.g., amino acid substitutions) and of nucleic acid vectors (e.g., substitutions or deletions of a D-sequence, and insertions of transcriptional regulator binding elements) can confer AAVrh74 particles with various beneficial properties, such as enhanced binding to particular cell types, enhanced interactions with cells and/or their biological machinery, enhanced transduction of cells, enhanced expression of a transgene within a cell, among other properties. Combinations of multiple modifications (e.g., combinations of various capsid protein modifications and/or nucleic acid vector modifications) can have synergistic effects on various properties of AAVrh74 particles in which they are incorporated. According to some aspects, modification of an AAV nucleic acid vector comprises modification of the left or right inverted terminal repeat (ITR) of the vector. In some embodiments, a modification of an AAV nucleic acid vector comprises substitution of the D-sequence in either the left or right ITR of the AAV vector. For example, in some embodiments a modification of an AAV nucleic acid vector comprises substitution of a sequence (e.g., the D-sequence in an ITR) in the AAV nucleic acid vector with another sequence (e.g., an S-sequence or a glucocorticoid receptor-binding element (GRE)). Substitution of a sequence (e.g., the D-sequence in an ITR) in an AAV nucleic acid vector with another sequence (e.g., an S-sequence or a GRE) can increase transduction efficiency and/or transgene expression levels of an AAV particle comprising the AAV nucleic acid vector. According to some aspects, a recombinant AAVrh74 particle disclosed herein comprises a capsid protein having one or more amino acid substitutions, in some embodiments in addition to an AAV nucleic acid vector which is modified. Encapsidation of a modified AAV nucleic acid vector in an AAVrh74 capsid comprising one or more amino acid substitutions can result in improved properties of the AAV particle comprising the modified AAV nucleic acid vector and the capsid comprising the one or more amino acid substitutions, in relation to a corresponding AAV particle that comprises an unmodified AAV nucleic acid vector and/or a capsid not comprising amino acid substitutions. In some embodiments, an improved property is an improvement of transduction efficiency, i.e., the efficiency of an AAV particle to deliver a genetic payload to a cell of interest.

According to some aspects of the present disclosure, capsid proteins are provided. In some embodiments, a capsid protein comprises an amino acid substitution at a position corresponding to Y447, T494, K547, N665, and/or Y733 of the wild-type AAVrh74 capsid protein of SEQ ID NO: 1, wherein the capsid protein is an AAVrh74 serotype capsid protein. In some embodiments, the substitution is Y447F, T494V, K547R, N665R, and/or Y733F.

According to some aspects, AAVrh74 particles are provided. In some embodiments, an AAVrh74 particle comprises a capsid protein disclosed herein. In some embodiments, an AAVrh74 particle further comprises a nucleic acid vector, wherein the nucleic acid vector comprises a first inverted terminal repeat (ITR) comprising a first D-sequence and a second ITR comprising a second D-sequence, wherein the first D-sequence or the second D-sequence is substituted with an S-sequence. In some embodiments, the S-sequence comprises, consists essentially of, or consists of the nucleotide sequence TATTAGATCTGATGGCCGCT (SEQ ID NO: 17).

In some embodiments, an AAVrh74 particle comprises a nucleic acid vector, wherein the nucleic acid vector comprises a first inverted terminal repeat (ITR) comprising a first D-sequence and a second ITR comprising a second D-sequence, wherein the first D-sequence and/or the second D-sequence is substituted with a glucocorticoid receptor-binding element (GRE). In some embodiments, the GRE comprises, consists essentially of, or consists of the nucleotide sequence AGAACANNNTGTTCT (SEQ ID NO: 18), or its reverse or reverse complement, wherein each N is independently a T, C, G, or A.

According to some aspects of the present disclosure, compositions comprising AAV capsid proteins or AAV particles are provided. In some embodiments, a composition disclosed herein comprises an AAVrh74 capsid protein disclosed herein. In some embodiments, a composition disclosed herein comprises an AAVrh74 particle disclosed herein.

According to some aspects, methods of contacting a cell are provided herein. In some embodiments, a method comprises contacting a cell with a composition comprising an AAVrh74 particle, wherein the AAVrh74 particle comprises a capsid protein and a nucleic acid vector,
(i) wherein the capsid protein comprises an amino acid substitution at a position corresponding to Y447, T494, K547, N665, and/or Y733 of the wild-type AAVrh74 capsid protein of SEQ ID NO: 1, optionally wherein the substitution is Y447F, T494V, K547R, N665R, and/or Y733F, and/or
(ii) wherein the nucleic acid vector comprises a first inverted terminal repeat (ITR) comprising a first D-sequence and a second ITR comprising a second D-sequence, wherein the first D-sequence or the second D-sequence is substituted with an S-sequence, optionally wherein the S-sequence comprises, consists essentially of, or consists of the nucleotide sequence TATTAGATCTGATGGCCGCT (SEQ ID NO: 17).

In some embodiments, a method comprises contacting a cell with a composition comprising an AAVrh74 particle, wherein the AAVrh74 particle comprises a capsid protein and a nucleic acid vector,
(i) wherein the capsid protein comprises an amino acid substitution at a position corresponding to Y447, T494, K547, N665, and/or Y733 of the wild-type AAVrh74 capsid protein of SEQ ID NO: 1, optionally wherein the substitution is Y447F, T494V, K547R, N665R, and/or Y733F, and/or
(ii) wherein the nucleic acid vector comprises a first inverted terminal repeat (ITR) comprising a first D-sequence and a second ITR comprising a second D-sequence, wherein the first D-sequence and/or the second D-sequence is substituted with a glucocorticoid receptor-binding element (GRE), optionally wherein the GRE comprises, consists essentially of, or consists of the nucleotide sequence AGAACANNNTGTTCT (SEQ ID NO: 18), or its reverse or reverse complement, wherein each N is independently a T, C, G, or A.

In some embodiments, the capsid protein comprises an amino acid substitution at a position corresponding to Y447, T494, K547, N665, and/or Y733 of the wild-type AAVrh74 capsid protein of SEQ ID NO: 1, optionally wherein the substitution is Y447F, T494V, K547R, N665R, and/or Y733F.

In some embodiments, the nucleic acid vector comprises the first ITR and the second ITR, wherein the first D-sequence or the second D-sequence is substituted with an S-sequence, optionally wherein the S-sequence comprises, consists essentially of, or consists of the nucleotide sequence TATTAGATCTGATGGCCGCT (SEQ ID NO: 17).

In some embodiments, the nucleic acid vector comprises the first ITR and the second ITR, wherein the first D-sequence and/or the second D-sequence is substituted with the GRE, optionally wherein the GRE comprises, consists essentially of, or consists of the nucleotide sequence AGAACANNNTGTTCT (SEQ ID NO: 18), or its reverse or reverse complement, wherein each N is independently a T, C, G, or A.

In some embodiments, the capsid protein comprises an amino acid substitution at a position corresponding to Y447, T494, K547, N665, and/or Y733 of the wild-type AAVrh74 capsid protein of SEQ ID NO: 1, and the nucleic acid vector comprises the first ITR and the second ITR, wherein the first D-sequence or the second D-sequence is substituted with the S-sequence, optionally wherein the substitution is Y447F, T494V, K547R, N665R, and/or Y733F, and optionally wherein the S-sequence comprises, consists essentially of, or consists of the nucleotide sequence TATTAGATCTGATGGCCGCT (SEQ ID NO: 17).

In some embodiments, the capsid protein comprises an amino acid substitution at a position corresponding to Y447, T494, K547, N665, and/or Y733 of the wild-type AAVrh74 capsid protein of SEQ ID NO: 1, and the nucleic acid vector comprises the first ITR and the second ITR, wherein the first D-sequence and/or the second D-sequence is substituted with the GRE, optionally wherein the substitution is Y447F, T494V, K547R, N665R, and/or Y733F, and optionally wherein the GRE comprises, consists essentially of, or consists of the nucleotide sequence AGAACANNNTGTTCT (SEQ ID NO: 18), or its reverse or reverse complement, wherein each N is independently a T, C, G, or A.

In some embodiments, the capsid protein comprises amino acid substitutions at positions corresponding to:
(a) Y447 and Y733, optionally wherein the substitutions are Y447F and Y733F;
(b) Y447, Y733, and N665, optionally wherein the substitutions are Y447F, Y733F, and N665R;
(c) Y447, Y733, and T494, optionally wherein the substitutions are Y447F, Y733F, and T494V;
(d) Y447, Y733, and K547, optionally wherein the substitutions are Y447F, Y733F, and K547R; or
(e) Y447, Y733, N665, T494, and K547, optionally wherein the substitutions are Y447F, Y733F, N665R, T494V, and K547R, of the wild-type AAVrh74 capsid protein of SEQ ID NO: 1.

In some embodiments, the first ITR and the second ITR are each an AAV2 serotype ITR or an AAV3 serotype ITR.

In some embodiments, the first D-sequence is substituted with the S-sequence, or the first D-sequence is substituted with the GRE. In some embodiments, the second D-sequence is substituted with the S-sequence, or the second D-sequence is substituted with the GRE. In some embodiments, the S-sequence comprises, consists essentially of, or consists of the nucleotide sequence TATTAGATCTGATGGCCGCT (SEQ ID NO: 17), or the GRE comprises, consists essentially of, or consists of the nucleotide sequence AGAACANNNTGTTCT (SEQ ID NO: 18), or its reverse or reverse complement, wherein each N is independently a T, C, G, or A.

In some embodiments, the transduction efficiency of the AAVrh74 particle is at least two-fold higher than a wild-type AAVrh74 particle. In some embodiments, the packaging efficiency of the AAVrh74 particle is decreased relative to a wild-type AAVrh74 particle.

In some embodiments, the composition further comprises a pharmaceutically-acceptable carrier.

In some embodiments, the cell is a mammalian cell. In some embodiments, the cell is a muscle cell. In some embodiments, the cell is a skeletal muscle cell. In some embodiments, the cell is a gastrocnemius cell or a tibialis anterior cell.

In some embodiments, the nucleic acid vector comprises a regulatory element. In some embodiments, the regulatory element comprises a promoter, an enhancer, a silencer, an insulator, a response element, an initiation site, a termination signal, or a ribosome binding site. In some embodiments, the promoter is a constitutive promoter. In some embodiments, the promoter is an inducible promoter. In some embodiments, the promoter is a tissue-specific promotor, a cell type-specific promoter, or a synthetic promoter.

In some embodiments, the nucleic vector comprises a nucleotide sequence of a gene of interest. In some embodiments, the gene of interest encodes a therapeutic protein or a diagnostic protein.

In some embodiments, the contacting is in vivo.

In some embodiments, the method further comprises administering the composition comprising the AAVrh74 particle to a subject.

In some embodiments, the cell is in the subject.

In some embodiments, the subject is human. In some embodiments, the subject is at risk of or suffering from a muscle disease, optionally wherein the muscle disease is amyotrophic lateral sclerosis, Charcot-Marie-Tooth disease, multiple sclerosis, muscular dystrophy, myasthenia gravis, myopathy, myositis, peripheral neuropathy, or spinal muscular atrophy. In some embodiments, the muscle disease is Duchenne muscular dystrophy, optionally wherein the subject has a mutation in a dystrophin gene. In some embodiments, the muscle disease is limb-girdle muscular dystrophy. In some embodiments, the muscle disease is X-linked myotubular myopathy, optionally wherein the subject has a mutation in a MTM1 gene.

In some embodiments, the composition is administered to the subject by subcutaneous injection, by intramuscular injection, by intravenous injection, by intraperitoneal injection, or orally.

In some embodiments, the contacting is in vitro or ex vivo.

### BRIEF DESCRIPTION OF DRAWINGS

FIGS. 1A-1B show transduction efficiency of wild-type (WT) and Y-F mutant ssAAVrh74 vectors in human HeLa (FIG. 1A) and mouse C2C12 (FIG. 1B) cells. Cells were transduced with each vector at the indicated vector genome copy numbers (vgs)/cell at 37° C. for 2 hours, and transgene expression was visualized under a fluorescence microscope 72 hours post-transduction. Data were quantitated using ImageJ software. The left panels show EGFP fluorescence in cells following transduction. The data in the right panel of FIG. 1A show quantification of transgene expression (pixels²/visual field) following transduction with 1,000 vgs/cell (left, lighter bars) or 3,000 vgs/cell (right, darker bars) for each of WT, Y733F, and Y447+733F ssAAVrh74 vectors. The data in the right panel of FIG. 1B show transgene expression (pixels²/visual field) following transduction with 3,000 vgs/cell (left, lighter bars) or 9,000 vgs/cell (right, darker bars) for each of WT, Y733F, and Y447+733F ssAAVrh74 vectors.

FIG. 2 shows transduction efficiency of wild-type (“WT”) and Y733+447F+T494V triple mutant (“TM”) ssAAVrh74 vectors in primary human skeletal muscle cells. Cells were transduced with each vector at the indicated multiplicity of infection (vgs/cell), and transgene expression levels were quantitated as described above in FIGS. 1A-1B. The left panel shows EGFP fluorescence in skeletal muscle cells following transduction. The right panel shows quantification of transgene expression (pixels²/visual field) following transduction with 1,000 vgs/cell (left, lighter bars) or 3,000 vgs/cell (right, darker bars) for WT and TM AAVrh74 vectors, respectively.

FIGS. 3A-3B show transduction efficiency of ssAAV-rh74 mutants in Hela cells. FIG. 3A shows GFP fluorescence 72 hours post-transduction with 3,000 vgs/cell of wild-type (WT) or capsid mutant ssAAVrh74 vectors. FIG. 3B shows quantitation of the GFP fluorescence transduction data (transgene expression, measured as pixels²/visual field).

FIGS. 4A-4C show transduction efficiency of wild-type (“WT”) ssAAVrh74 vectors or ssAAVrh74 vectors in which the D-sequence of the left ITR (“LC1”) or of the right ITR (“LC2) was substituted. FIG. 4A shows transgene expression mediated by WT, LC1, or LC2 ssAAVrh74 vectors in Hela cells. The left panel shows hrGFP fluorescence in HeLa cells following transduction with 1,000 vgs/cell, 3,000 vgs/cell, or 10,000 vgs/cell of each respective ssAAVrh74 vector. The right panel shows quantification of transgene expression (pixels²/visual field) following transduction with 1,000 vgs/cell (left bar of each set of bars), 3,000 vgs/cell (middle bar), or 10,000 vgs/cell (right bar) of WT, LC1, or LC2 AAVrh74 vector, respectively. FIG. 4B shows vector genome copy numbers (copy number per μg of DNA×10⁸) in Hela cells transduced with WT, LC1, or LC2 ssAAVrh74 vectors. Each set of three bars shows the copy number following transduction with 1,000 vgs/cell (left bar), 3,000 vgs/cell (middle bar), or 10,000 vgs/cell (right bar). FIG. 4C shows transgene expression mediated by WT, LC1, or LC2 ssAAVrh74 vectors in primary human skeletal muscle cells. The left panel shows hrGFP fluorescence in primary human skeletal muscle cells following transduction with 1,000 vgs/cell, 3,000 vgs/cell, or 10,000 vgs/cell of each respective ssAAVrh74 vector. The right panel shows quantification of transgene expression (pixels²/visual field) following transduction with 1,000 vgs/cell (left bar of each set of bars), 3,000 vgs/cell (middle bar), or 10,000 vgs/cell (right bar) for WT, LC1, or LC2 AAVrh74 vectors, respectively. For both FIG. 3A and FIG. 3B, cells were transduced with each vector at the indicated multiplicity of infection (vgs/cell) at 37° C. for 2 hours, and transgene expression was visualized under a fluorescence microscope 72 hours post-transduction. Data were quantitated using the ImageJ software.

FIG. 5 shows transduction efficiency of HeLa cells using wild-type (“WT”) ssAAVrh74 vector, Y447+733F+T494V triple mutant (“TM”) ssAAVrh74 vector, and Y447+733F+T494V triple mutant ssAAVrh74 vector with additional substitution of the D-sequence of the left ITR (“Opt^{X}”). HeLa cells were transduced with 1,000 vgs/cell and transduction efficiency was determined 72 hours post-transduction.

FIGS. 6A-6B show transduction efficiency of WT, TM, and Opt^{X} ssAAV-rh74 vectors in Hela cells, measured by flow cytometry quantification of GFP fluorescence (FIG. 6A) and quantification of flow cytometry mean GFP fluorescence (FIG. 6B). WT, TM, and Opt^{X} are as defined in FIG. 5 above. HeLa cells were transduced with 1,000 vgs/cell and transduction efficiency was determined 72 hours post-transduction.

FIGS. 7A-7D show efficacy of WT and Opt^{X} ssAAVrh74 vectors in vivo following intravenous administration of 1×10¹² vgs/mouse in C57B16 mice. FIG. 7A shows transgene expression in gastrocnemius (GA) muscle, and FIG. 7B shows transgene expression in tibialis anterior (TA) muscle quantified after intravenous administration of the vectors. FIG. 7C shows vector genome copy numbers quantified in various tissues harvested 8 weeks following administration of the vectors. FIG. 7D shows relative transgene expression measured in liver, GA, and TA following administration of the vectors. Transgene expression data were quantified using NIH ImageJ software analysis of fluorescence microscopy images.

FIGS. 8A-8D show efficacy of WT, GenX, and GenY vectors in vitro. FIG. 8A shows schematic structures of the WT (with D-sequences at the ITR ends distal from the termini of the nucleic acid vector), GenX (with one D-sequence substituted), and GenY (with a portion of one D-sequence substituted with a GRE) genomes. FIG. 8B shows the transduction efficiency of GenX and GenY AAVrh74 vectors in mouse C2C12 cells in the absence or presence of tyrphostin (“Tyr.”). FIG. 8C shows transduction efficiency of WT, GenX, and GenY AAVrh74 vectors in primary human skeletal muscle cells. Cells were transduced with each vector at the indicated vector genome copy number per cell at 37° C. for 2 hours, and transgene expression was visualized under a fluorescence microscope 72 hours post-transduction. Transgene expression was quantified using NIH ImageJ software analysis of fluorescence microscopy images. FIG. 8D shows vector genome copy numbers quantified in primary human skeletal muscle cells transduced with WT, GenX, and GenY AAVrh74 vectors.

FIGS. 9A-9B show the efficacy of Opt^{X} AAVrh74 vectors. FIG. 9A shows reverse transcription-quantitative PCR (RT-qPCR) measurements of hrGFP mRNA copy number per μg of total RNA extracted from liver, diaphragm, and heart tissues of mice administered PBS, wild-type AAVrh74 particles containing an hrGFP transgene (“WT”) or Opt^{X} AAVrh74 particles containing an hrGFP transgene (“Opt^{X}”). FIG. 9B shows relative expression levels of hrGFP in liver, diaphragm, and heart tissue samples from mice administered WT or Opt^{X} AAVrh74 particles containing an hrGFP transgene.

FIGS. 10A-10B show control measurements of gene expression in liver, diaphragm, and heart tissues of mice administered PBS, WT or Opt^{X} AAVrh74 particles containing an hrGFP transgene. FIG. 10A shows expression of β-actin measured by RT-qPCR. FIG. 10B shows cycle threshold (CT) values from β-actin RT-qPCR measurement.

FIGS. 11A-11B show the efficacy of Opt^{Y} AAVrh74 vectors. FIG. 11A shows fluorescence microscopy images of liver, gastrocnemius (“GA”), and tibialis anterior (“TA”) tissue sections from mice administered Y447+733F+T494V triple mutant AAVrh74 particles containing an hrGFP transgene (“TM”) or Opt^{Y} AAVrh74 particles containing an hrGFP transgene (“Opt^{Y}”). FIG. 11B shows quantification of hrGFP transgene expression from fluorescence microscopy images.

FIG. 12 shows quantification of vector genome copy number in liver, heart, diaphragm, gastrocnemius (“GA muscle”) and tibialis anterior (“TA muscle”) tissues of mice administered Y447+733F+T494V triple mutant AAVrh74 particles containing an hrGFP transgene (“TM”) or Opt^{Y} AAVrh74 particles containing an hrGFP transgene (“Opt^{Y}”).

FIG. 13 shows quantification of hrGFP mRNA expression per vector genome copy number in liver, heart, diaphragm, gastrocnemius (“GA muscle”) and tibialis anterior (“TA muscle”) tissues of mice administered Y447+733F+T494V triple mutant AAVrh74 particles containing an hrGFP transgene (“TM”) or Opt^{Y} AAVrh74 particles containing an hrGFP transgene (“Opt^{Y}”).

### DETAILED DESCRIPTION

The present disclosure is based at least in part on the development of adeno-associated virus (AAV) capsid proteins, particles, genomes, nucleic acid vectors, and plasmids useful in the delivery of various cargoes to particular cells, facilitating transgene expression therein. The disclosure relates, at least in part, to the finding that incorporation of amino acid substitutions in AAVrh74 capsid proteins and/or nucleotide sequence modifications (e.g., substitutions or deletions) in AAV nucleic acid vectors results in improved transduction efficiency and/or transgene expression. The AAV capsid proteins, particles, genomes, nucleic acid vectors, and plasmids disclosed herein may be used in a variety of applications including but not limited to compositions and methods (e.g., therapeutic methods). Therapeutic methods disclosed herein include those useful in the treatment of diseases (e.g., muscular disorders, such as muscular dystrophies), in subjects in need thereof.

Provided herein are compositions, including AAV capsid proteins, AAV particles, nucleic acids comprised within AAV particles, which nucleic acids that comprise one or more modifications in one or more ITRs, and methods of using the compositions for transducing a cell of interest (e.g., for treating a disease or condition in a subject).

### Capsid Proteins

Provided herein is an AAV capsid protein having one or more mutations characterized by amino acid substitutions. In some embodiments, an AAV capsid protein disclosed herein comprises an amino acid substitution at one or more positions corresponding to Y447, T494, K547, N665, or Y733 of the wild-type AAVrh74 capsid protein of SEQ ID NO: 1. In some embodiments, the amino acid substitutions are selected from Y447F, T494V, K547R, N665R, and/or Y733F. In some embodiments, an AAV capsid protein disclosed herein comprises amino acid substitutions at positions corresponding to Y447 and Y733; Y447, Y733, and N665; Y447, Y733, and T494; Y447, Y733, and K547; or Y447, Y733, N665, T494, and K547 of the wild-type AAVrh74 capsid protein of SEQ ID NO: 1. In some embodiments, an AAV capsid protein disclosed herein comprises amino acid substitutions at positions corresponding to Y447F and Y733F; Y447F, Y733F, and N665R; Y447F, Y733F, and T494V; Y447F, Y733F, and K547R; or Y447F, Y733F, N665R, T494V, and K547R of the wild-type AAVrh74 capsid protein of SEQ ID NO: 1.

In some embodiments, an AAV capsid protein as disclosed herein is a VP1 protein, a VP2 protein, or a VP3 protein. The VP1, VP2, and VP3 capsid proteins are each encoded from the same segment of the AAV genome, and differ in their N termini based on alternative mRNA splicing.

| Example of an amino acid sequence of AAVrh74 capsid protein: | |
|---|---|
| (SEQ ID NO: 1) | |
| 1 | MAADGYLPDW LEDNLSEGIR EWWDLKPGAP KPKANQQKOD NGRGLVLPGY |
| 51 | KYLGPFNGLD KGEPVNAADA AALEHDKAYD QQLQAGDNPY LRYNHADAEF |
| 101 | QERLQEDTSF GGNLGRAVFQ AKKRVLEPLG LVESPVKTAP GKKRPVEPSP |
| 151 | QRSPDSSTGI GKKGQQPAKK RLNFGQTGDS ESVPDPQPIG EPPAGPSGLG |
| 201 | SGTMAAGGGA PMADNNEGAD GVGSSSGNWH CDSTWLGDRV ITTSTRTWAL |
| 251 | PTYNNHLYKQ ISNGTSGGST NDNTYFGYST PWGYFDFNRF HCHFSPRDWQ |
| 301 | RLINNNWGFR PKRLNFKLEN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRTGN NFEFSYNFED VPFHSSYAHS QSLDRLMNPL IDQYLYYLSR |
| 451 | TQSTGGTAGT QQLLFSQAGP NNMSAQAKNW LPGPCYRQQR VSTILSQNNN |
| 501 | SNFAWTGATK YHLNGRDSLV NPGVAMATHK DDEERFFPSS GVLMFGKOGA |
| 551 | GKDNVDYSSV MLISEEEIKT TNPVATEQYG VVADNLQQON AAPIVGAVNS |
| 601 | QGALPGMVWQ NRDVYLOGPI WAKIPHTDGN FHPSPLMGGF GLKHPPPQIL |
| 651 | IKNTPVPADP PTTFNQAKLA SFITQYSTGQ VSVEIEWELQ KENSKRWNPE |
| 701 | IQYTSNYYKS TNVDFAVNTE GTYSEPRPIG TRYLTRNL |

Example of a nucleotide sequence encoding AAVrh74 capsid protein:
(SEQ ID NO: 22)
atggctgccgatggttatcttccagattggctcgaggacaacctctctgagggcattcgcgagtggtgggacctga
aacctggagccccgaaacccaaagccaaccagcaaaagcaggacaacggccggggtctggtgcttcctggctacaa
gtacctcggacccttcaacggactcgacaagggggagcccgtcaacgcggcggacgcagcggccctcgagcacgac
aaggcctacgaccagcagctccaaggggtgacaatccgtacctgcggtataatcacgccgacgccgagtttcagg
agcgtctgcaagaagatacgtcttttgggggcaacctcgggcgcgcagtcttccaggccaaaaagcgggttctcga
acctctgggcctggttgaatcgccggttaagacggctcctggaaagaagagaccggtagagccatcaccccagcgc
tctccagactcctctacgggcatcggcaagaaaggccagcagcccgcaaaaaagagactcaattttgggcagactg
gcgactcagagtcagtccccgaccctcaaccaatcggagaaccaccagcaggcccctctggtctgggatctggtac
aatggctgcaggcggtggcgctccaatggcagacaataacgaaggcgccgacggagtgggtagttcctcaggaaat
tggcattgcgattccacatggctgggcgacagagtcatcaccaccagcacccgcacctgggccctgcccacctaca
acaaccacctctacaagcaaatctccaacgggacctcgggaggaagcaccaacgacaacacctacttcggctacag
caccccctgggggtattttgacttcaacagattccactgccacttttcaccacgtgactggcagcgactcatcaac
aacaactggggattccggcccaagaggctcaacttcaagctcttcaacatccaagtcaaggaggtcacgcagaatg
aaggcaccaagaccatcgccaataaccttaccagcacgattcaggtctttacggactcggaataccagctcccgta
cgtgctcggctcggcgcaccagggctgcctgcctccgttcccggcggacgtcttcatgattcctcagtacgggtac
ctgactctgaacaatggcagtcaggctgtgggccggtcgtccttctactgcctggagtactttccttctcaaatgc
tgagaacgggcaacaactttgaattcagctacaacttcgaggacgtgcccttccacagcagctacgcgcacagcca
gagcctggaccggctgatgaaccctctcatcgaccagtacttgtactacctgtcccggactcaaagcacgggcggt
actgcaggaactcagcagttgctattttctcaggccgggcctaacaacatgtcggctcaggccaagaactggctac
ccggtccctgctaccggcagcaacgcgtctccacgacactgtcgcagaacaacaacagcaactttgcctggacggg
tgccaccaagtatcatctgaatggcagagactctctggtgaatcctggcgttgccatggctacccacaaggacgac
gaagagcgattttttccatccagcggagtcttaatgtttgggaaacagggagctggaaaagacaacgtggactata
gcagcgtgatgctaaccagcgaggaagaaataaagaccaccaacccagtggccacagaacagtacggcgtggtggc
cgataacctgcaacagcaaaacgccgctcctattgtaggggccgtcaatagtcaaggagccttacctggcatggtg
tggcagaaccgggacgtgtacctgcagggtcccatctgggccaagattcctcatacggacggcaactttcatccct
cgccgctgatgggaggctttggactgaagcatccgcctcctcagatcctgattaaaaacacacctgttcccgccga
tcctccgaccaccttcaatcaggccaagctggcttctttcatcacgcagtacagtaccggtcaggtcagcgtggag
atcgagtgggagctgcagaaggagaacagcaaacgctggaacccagagattcagtacacttccaactactacaaat
ctacaaatgtggactttgctgtcaatactgagggtacttattccgagcctcgccccattggcacccgttacctcac
ccgtaatctgtaa

The different capsid proteins VP1, VP2, and VP3 are defined according to numbering of the full-length VP1 protein. In some embodiments, for AAVrh74 capsid proteins, a VP1 capsid protein is defined by amino acids 1-738 of SEQ ID NO: 1; a VP2 capsid protein is defined by amino acids 138-738 of SEQ ID NO: 1; and a VP3 capsid protein is defined by amino acids 204-738 of SEQ ID NO: 1. Numbering of AAV capsid proteins is provided according to the VP1 sequence. For example, Y447 refers to the tyrosine at position 447 of SEQ ID NO: 1 in a VP1 protein or the corresponding tyrosine in a VP2 or VP3 protein. Similarly, T494, K547, N665, and Y733 refer to the threonine at position 494, lysine at position 547, asparagine at position 665, and tyrosine at position 733 of SEQ ID NO: 1, respectively, in a VP1 protein, or the corresponding amino acids in a VP2 or VP3 protein.

An AAV capsid protein disclosed herein can be of any serotype, or can be a chimeric capsid protein (i.e., comprising segments from capsid proteins of two or more serotypes). In some embodiments, a capsid protein disclosed herein is an AAV1, AAV2, AAV3, AAV4, AAV5, AAV6, AAV7, AAV8, AAV9, AAV10, AAV11, AAV12, AAV13, AAVrh10, or AAVrh74 capsid protein. In some embodiments, an AAV capsid protein as provided herein is of serotype rh74. Amino acid sequences of capsid proteins of other AAV serotypes are known and can be aligned with SEQ ID NO: 1 (AAVrh74 capsid protein) using techniques known in the art. Examples of amino acid sequences of AAV capsid proteins of various serotypes are provided below:

| Example of wild-type AAV1 capsid protein | |
|---|---|
| (SEQ ID NO: 2) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTINDGV TTIANNLIST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEEVP FHSSYAHSQS LDRLMNPLID QYLYYLNRTQ |
| 451 | NQSGSAQNKD LLFSRGSPAG MSVQPKNWLP GPCYRQQRVS KTKTDNNNSN |
| 501 | FTWIGASKYN LNGRESIINP GTAMASHKDD EDKFFPMSGV MIFGKESAGA |
| 551 | SNTALDNVMI TDEEEIKATN PVATERFGTV AVNFQSSSTD PATGDVHAMG |
| 601 | ALPGMVWQDR DVYLQGPIWA KIPHTDGHFH PSPLMGGFGL KNPPPQILIK |
| 651 | NTPVPANPPA EFSATKFASF ITQYSTGQVS VEIEWELQKE NSKRWNPEVQ |
| 701 | YTSNYAKSAN VDFTVDNNGL YTEPRPIGTR YLTRPL |
| Example of wild-type AAV2 capsid protein | |
| (SEQ ID NO: 3) | |
| 1 | MAADGYLPDW LEDTLSEGIR QWWKLKPGPP PPKPAERHKD DSRGLVLPGY |
| 51 | KYLGPFNGLD KGEPVNEADA AALEHDKAYD RQLDSGDNPY LKYNHADAEF |
| 101 | QERLKEDTSF GGNLGRAVFQ AKKRVLEPLG LVEEPVKTAP GKKRPVEHSP |
| 151 | VEPDSSSGTG KAGQQPARKR LNFGQTGDAD SVPDPQPLGQ PPAAPSGLGT |
| 201 | NTMATGSGAP MADNNEGADG VGNSSGNWHC DSTWMGDRVI TISTRTWALP |
| 251 | TYNNHLYKQI SSQSGASNDN HYFGYSTPWG YFDFNRFHCH FSPRDWQRLI |
| 301 | NNNWGFRPKR LNFKLFNIQV KEVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFT FSYTFEDVPF HSSYAHSQSL DRLMNPLIDQ YLYYLSRINT |
| 451 | PSGTTTQSRL QFSQAGASDI RDQSRNWLPG PCYRQQRVSK TSADNNNSEY |
| 501 | SWIGATKYHL NGRDSLVNPG PAMASHKDDE EKFFPQSGVL IFGKQGSEKT |
| 551 | NVDIEKVMIT DEEEIRTTNP VATEQYGSVS TNLQRGNRQA ATADVNTQGV |
| 601 | LPGMVWQDRD VYLQGPIWAK IPHTDGHFHP SPLMGGFGLK HPPPQILIKN |
| 651 | TPVPANPSTT FSAAKFASFI TQYSTGQVSV EIEWELQKEN SKRWNPEIQY |
| 701 | TSNYNKSVNV DFTVDINGVY SEPRPIGTRY LTRNL |
| Example of wild-type AAV3 capsid protein | |
| (SEQ ID NO: 4) | |
| 1 | MAADGYLPDW LEDNLSEGIR EWWALKPGVP QPKANQQHQD NRRGLVLPGY |
| 51 | KYLGPGNGLD KGEPVNEADA AALEHDKAYD QQLKAGDNPY LKYNHADAEF |
| 101 | QERLQEDTSF GGNLGRAVFQ AKKRILEPLG LVEEAAKTAP GKKGAVDQSP |
| 151 | QEPDSSSGVG KSGKQPARKR LNFGQTGDSE SVPDPQPLGE PPAAPTSLGS |
| 201 | NTMASGGGAP MADNNEGADG VGNSSGNWHC DSQWLGDRVI TTSTRTWALP |
| 251 | TYNNHLYKQI SSQSGASNDN HYFGYSTPWG YFDFNRFHCH FSPRDWQRLI |
| 301 | NNNWGFRPKK LSFKLFNIQV RGVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFQ FSYTFEDVPF HSSYAHSQSL DRLMNPLIDQ YLYYLNRTQG |
| 451 | TTSGTTNQSR LLFSQAGPQS MSLQARNWLP GPCYRQQRLS KTANDNNNSN |
| 501 | FPWTAASKYH LNGRDSLVNP GPAMASHKDD EEKFFPMHGN LIFGKEGTTA |
| 551 | SNAELDNVMI TDEEEIRTTN PVATEQYGTV ANNLQSSNTA PTTGTVNHQG |
| 601 | ALPGMVWQDR DVYLQGPIWA KIPHTDGHFH PSPLMGGFGL KHPPPQIMIK |
| 651 | NTPVPANPPT TFSPAKFASF ITQYSTGQVS VEIEWELQKE NSKRWNPEIQ |
| 701 | YTSNYNKSVN VDFTVDINGV YSEPRPIGTR YLTRNL |
| Example of wild-type AAV4 capsid protein | |
| (SEQ ID NO: 5) | |
| 1 | MTDGYLPDWL EDNLSEGVRE WWALQPGAPK PKANQQHQDN ARGLVLPGYK |
| 51 | YLGPGNGLDK GEPVNAADAA ALEHDKAYDQ QLKAGDNPYL KYNHADAEFQ |
| 101 | QRLQGDTSFG GNLGRAVFQA KKRVLEPLGL VEQAGETAPG KKRPLIESPQ |
| 151 | QPDSSTGIGK KGKQPAKKKL VFEDETGAGD GPPEGSTSGA MSDDSEMRAA |
| 201 | AGGAAVEGGQ GADGVGNASG DWHCDSTWSE GHVITTSTRT WVLPTYNNHL |
| 251 | YKRLGESLQS NTYNGFSTPW GYFDFNRFHC HFSPRDWQRL INNNWGMRPK |
| 301 | AMRVKIFNIQ VKEVTTSNGE TTVANNLIST VQIFADSSYE LPYVMDAGQE |
| 351 | GSLPPFPNDV FMVPQYGYCG LVTGNISQQQ TDRNAFYCLE YFPSQMLRTG |
| 401 | NNFEITYSFE KVPFHSMYAH SQSLDRLMNP LIDQYLWGLQ STTTGTTLNA |
| 451 | GTATTNFTKL RPTNFSNFKK NWLPGPSIKQ QGFSKTANQN YKIPATGSDS |
| 501 | LIKYETHSTL DGRWSALTPG PPMATAGPAD SKFSNSQLIF AGPKQNGNTA |
| 551 | TVPGTLIFTS EEELAATNAT DTDMWGNLPG GDQSNSNLPT VDRLTALGAV |
| 601 | PGMVWQNRDI YYQGPIWAKI PHTDGHFHPS PLIGGFGLKH PPPQIFIKNT |
| 651 | PVPANPATTF SSTPVNSFIT QYSTGQVSVQ IDWEIQKERS KRWNPEVQFT |
| 701 | SNYGQQNSLL WAPDAAGKYT EPRAIGTRYL THHL |
| Example of wild-type AAV5 capsid protein | |
| (SEQ ID NO: 6) | |
| 1 | MSFVDHPPDW LEEVGEGLRE FLGLEAGPPK PKPNQQHQDQ ARGLVLPGYN |
| 51 | YLGPGNGLDR GEPVNRADEV AREHDISYNE QLEAGDNPYL KYNHADAEFQ |
| 101 | EKLADDTSFG GNLGKAVFQA KKRVLEPFGL VEEGAKTAPT GKRIDDHFPK |
| 151 | RKKARTEEDS KPSTSSDAEA GPSGSQQLQI PAQPASSLGA DTMSAGGGGP |
| 201 | LGDNNQGADG VGNASGDWHC DSTWMGDRVV TKSTRTWVLP SYNNHQYREI |
| 251 | KSGSVDGSNA NAYFGYSTPW GYFDFNRFHS HWSPRDWQRL INNYWGFRPR |
| 301 | SLRVKIFNIQ VKEVTVQDST TTIANNLIST VQVFTDDDYQ LPYVVGNGTE |
| 351 | GCLPAFPPQV FTLPQYGYAT LNRDNTENPT ERSSFFCLEY FPSKMLRTGN |
| 401 | NFEFTYNFEE VPFHSSFAPS QNLFKLANPL VDQYLYRFVS TNNTGGVQFN |
| 451 | KNLAGRYANT YKNWFPGPMG RTQGWNLGSG VNRASVSAFA TTNRMELEGA |
| 501 | SYQVPPQPNG MTNNLQGSNT YALENTMIFN SQPANPGTTA TYLEGNMLIT |
| 551 | SESETQPVNR VAYNVGGQMA INNQSSTTAP ATGTYNLQEI VPGSVWMERD |
| 601 | VYLQGPIWAK IPETGAHFHP SPAMGGFGLK HPPPMMLIKN TPVPGNITSF |
| 651 | SDVPVSSFIT QYSTGQVIVE MEWELKKENS KRWNPEIQYT NNYNDPQFVD |
| 701 | FAPDSTGEYR TTRPIGTRYL TRPL |
| Example of wild-type AAV6 capsid protein | |
| (SEQ ID NO: 7) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPFG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TISTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTINDGV TTIANNLIST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEDVP FHSSYAHSQS LDRLMNPLID QYLYYLNRTQ |
| 451 | NQSGSAQNKD LLFSRGSPAG MSVQPKNWLP GPCYRQQRVS KTKTDNNNSN |
| 501 | FTWTGASKYN LNGRESIINP GTAMASHKDD KDKFFPMSGV MIFGKESAGA |
| 551 | SNTALDNVMI TDEEEIKATN PVATERFGTV AVNLQSSSTD PATGDVHVMG |
| 601 | ALPGMVWQDR DVYLQGPIWA KIPHTDGHFH PSPLMGGFGL KHPPPQILIK |
| 651 | NTPVPANPPA EFSATKFASF ITQYSTGQVS VEIEWELQKE NSKRWNPEVQ |
| 701 | YTSNYAKSAN VDFTVDNNGL YTEPRPIGTR YLTRPL |
| Example of wild-type AAV7 capsid protein | |
| (SEQ ID NO: 8) | |
| 1 | MAADGYLPDW LEDNLSEGIR EWWDLKPGAP KPKANQQKQD NGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP AKKRPVEPSP |
| 151 | QRSPDSSTGI GKKGQQPARK RLNFGQTGDS ESVPDPQPLG EPPAAPSSVG |
| 201 | SGTVAAGGGA PMADNNEGAD GVGNASGNWH CDSTWLGDRV ITTSTRTWAL |
| 251 | PTYNNHLYKQ ISSETAGSIN DNTYFGYSTP WGYFDFNRFH CHFSPRDWQR |
| 301 | LINNNWGFRP KKLRFKLFNI QVKEVTINDG VTTIANNLTS TIQVFSDSEY |
| 351 | QLPYVLGSAH QGCLPPFPAD VFMIPQYGYL TLNNGSQSVG RSSFYCLEYF |
| 401 | PSQMLRIGNN FEFSYSFEDV PFHSSYAHSQ SLDRLMNPLI DQYLYYLART |
| 451 | QSNPGGTAGN RELQFYQGGP STMAEQAKNW LPGPCFRQQR VSKILDQNNN |
| 501 | SNFAWIGATK YHLNGRNSLV NPGVAMATHK DDEDRFFPSS GVLIFGKTGA |
| 551 | TNKTTLENVL MTNEEEIRPT NPVATEEYGI VSSNLQAANT AAQTQVVNNQ |
| 601 | GALPGMVWQN RDVYLQGPIW AKIPHTDGNF HPSPLMGGFG LKHPPPQILI |
| 651 | KNIPVPANPP EVFTPAKFAS FITQYSTGQV SVEIEWELQK ENSKRWNPEI |
| 701 | QYTSNFEKQT GVDFAVDSQG VYSEPRPIGT RYLTRNL |
| Example of wild-type AAV8 protein | |
| (SEQ ID NO: 9) | |
| 1 | MAADGYLPDW LEDNLSEGIR EWWALKPGAP KPKANQQKQD DGRGLVLPGY |
| 51 | KYLGPFNGLD KGEPVNAADA AALEHDKAYD QQLQAGDNPY LRYNHADAEF |
| 101 | QERLQEDTSF GGNLGRAVFQ AKKRVLEPLG LVEEGAKTAP GKKRPVEPSP |
| 151 | QRSPDSSTGI GKKGQQPARK RLNFGQTGDS ESVPDPQPLG EPPAAPSGVG |
| 201 | PNTMAAGGGA PMADNNEGAD GVGSSSGNWH CDSTWLGDRV ITTSTRTWAL |
| 251 | PTYNNHLYKQ ISNGTSGGAT NDNTYFGYST PWGYFDFNRF HCHFSPRDWQ |
| 301 | RLINNNWGFR PKRLSFKLEN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRTGN NFQFTYTFED VPFHSSYAHS QSLDRLMNPL IDQYLYYLSR |
| 451 | TQTTGGTANT QTLGFSQGGP NTMANQAKNW LPGPCYRQQR VSTTTGQNNN |
| 501 | SNFAWTAGTK YHLNGRNSLA NPGIAMATHK DDEERFFPSN GILIFGKQNA |
| 551 | ARDNADYSDV MLTSEEEIKT TNPVATEEYG IVADNLQQQN TAPQIGTVNS |
| 601 | QGALPGMVWQ NRDVYLQGPI WAKIPHTDGN FHPSPLMGGF GLKHPPPQIL |
| 651 | IKNTPVPADP PTTFNQSKLN SFITQYSTGQ VSVEIEWELQ KENSKRWNPE |
| 701 | IQYTSNYYKS TSVDFAVNTE GVYSEPRPIG TRYLTRNL |
| Example of wild-type AAV9 capsid protein | |
| (SEQ ID NO: 10) | |
| 1 | MAADGYLPDW LEDNLSEGIR EWWALKPGAP QPKANQQHQD NARGLVLPGY |
| 51 | KYLGPGNGLD KGEPVNAADA AALEHDKAYD QQLKAGDNPY LKYNHADAEF |
| 101 | QERLKEDTSF GGNLGRAVFQ AKKRLLEPLG LVEEAAKTAP GKKRPVEQSP |
| 151 | QEPDSSAGIG KSGAQPAKKR LNFGQTGDTE SVPDPQPIGE PPAAPSGVGS |
| 201 | LTMASGGGAP VADNNEGADG VGSSSGNWHC DSQWLGDRVI TTSTRTWALP |
| 251 | TYNNHLYKQI SNSTSGGSSN DNAYFGYSTP WGYFDFNRFH CHFSPRDWQR |
| 301 | LINNNWGFRP KRLNFKLFNI QVKEVTDNNG VKTIANNLTS TVQVFTDSDY |
| 351 | QLPYVLGSAH EGCLPPFPAD VFMIPQYGYL TLNDGSQAVG RSSFYCLEYF |
| 401 | PSQMLRTGNN FQFSYEFENV PFHSSYAHSQ SLDRLMNPLI DQYLYYLSKT |
| 451 | INGSGQNQQT LKFSVAGPSN MAVQGRNYIP GPSYRQQRVS TTVTQNNNSE |
| 501 | FAWPGASSWA LNGRNSLMNP GPAMASHKEG EDRFFPLSGS LIFGKQGTGR |
| 551 | DNVDADKVMI TNEEEIKTIN PVATESYGQV ATNHQSAQAQ AQTGWVQNQG |
| 601 | ILPGMVWQDR DVYLQGPIWA KIPHTDGNFH PSPLMGGFGM KHPPPQILIK |
| 651 | NTPVPADPPT AFNKDKLNSF ITQYSTGQVS VEIEWELQKE NSKRWNPEIQ |
| 701 | YTSNYYKSNN VEFAVNTEGV YSEPRPIGTR YLTRNL |
| Example of wild-type AAV10 capsid protein | |
| (SEQ ID NO: 11) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPVEPSP |
| 151 | QRSPDSSTGI GKKGQQPAKK RLNFGQTGDS ESVPDPQPIG EPPAGPSGLG |
| 201 | SGTMAAGGGA PMADNNEGAD GVGSSSGNWH CDSTWLGDRV ITTSTRTWAL |
| 251 | PTYNNHLYKQ ISNGTSGGST NDNTYFGYST PWGYFDFNRF HCHFSPRDWQ |
| 301 | RLINNNWGFR PKRLNFKLEN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRIGN NFEFSYQFED VPFHSSYAHS QSLDRLMNPL IDQYLYYLSR |
| 451 | TQSTGGTAGT QQLLFSQAGP NNMSAQAKNW LPGPCYRQQR VSTTLSQNNN |
| 501 | SNFAWTGATK YHLNGRDSLV NPGVAMATHK DDEERFFPSS GVLMFGKQGA |
| 551 | GKDNVDYSSV MLTSEEEIKT TNPVATEQYG VVADNLQQQN AAPIVGAVNS |
| 601 | QGALPGMVWQ NRDVYLQGPI WAKIPHTDGN FHPSPLMGGF GLKHPPPQIL |
| 651 | IKNTPVPADP PTTFSQAKLA SFITQYSTGQ VSVEIEWELQ KENSKRWNPE |
| 701 | IQYTSNYYKS TNVDFAVNTD GTYSEPRPIG TRYLTRNL |
| Example of wild-type AAV11 capsid protein | |
| (SEQ ID NO: 23) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPLESPQ |
| 151 | EPDSSSGIGK KGKQPARKRL NFEEDTGAGD GPPEGSDTSA MSSDIEMRAA |
| 201 | PGGNAVDAGQ GSDGVGNASG DWHCDSTWSE GKVTTTSTRI WVLPTYNNHL |
| 251 | YLRLGTTSSS NTYNGFSTPW GYFDFNRFHC HFSPRDWQRL INNNWGLRPK |
| 301 | AMRVKIFNIQ VKEVITSNGE TTVANNLIST VQIFADSSYE LPYVMDAGQE |
| 351 | GSLPPFPNDV FMVPQYGYCG IVTGENQNQT DRNAFYCLEY FPSQMLRTGN |
| 401 | NFEMAYNFEK VPFHSMYAHS QSLDRLMNPL LDQYLWHLQS TTSGETLNQG |
| 451 | NAATTFGKIR SGDFAFYRKN WLPGPCVKQQ RESKTASQNY KIPASGGNAL |
| 501 | LKYDTHYTLN NRWSNIAPGP PMATAGPSDG DFSNAQLIFP GPSVTGNTTT |
| 551 | SANNLLFTSE EEIAATNPRD TDMFGQIADN NQNATTAPIT GNVTAMGVLP |
| 601 | GMVWQNRDIY YQGPIWAKIP HADGHFHPSP LIGGFGLKHP PPQIFIKNTP |
| 651 | VPANPATTFT AARVDSFITQ YSTGQVAVQI EWEIEKERSK RWNPEVQFTS |
| 701 | NYGNQSSMLW APDTTGKYTE PRVIGSRYLT NHL |
| Example of wild-type AAV12 protein | |
| (SEQ ID NO: 24) | |
| 1 | MAADGYLPDW LEDNLSEGIR EWWALKPGAP QPKANQQHQD NGRGLVLPGY |
| 51 | KYLGPFNGLD KGEPVNEADA AALEHDKAYD KQLEQGDNPY LKYNHADAEF |
| 101 | QQRLATDTSF GGNLGRAVFQ AKKRILEPLG LVEEGVKTAP GKKRPLEKTP |
| 151 | NRPTNPDSGK APAKKKQKDG EPADSARRIL DFEDSGAGDG PPEGSSSGEM |
| 201 | SHDAEMRAAP GGNAVEAGQG ADGVGNASGD WHCDSTWSEG RVTTTSTRTW |
| 251 | VLPTYNNHLY LRIGTTANSN TYNGFSTPWG YFDFNRFHCH FSPRDWQRLI |
| 301 | NNNWGLRPKS MRVKIFNIQV KEVTTSNGET TVANNLTSTV QIFADSTYEL |
| 351 | PYVMDAGQEG SFPPFPNDVF MVPQYGYCGV VTGKNQNQTD RNAFYCLEYF |
| 401 | PSQMLRTGNN FEVSYQFEKV PFHSMYAHSQ SLDRMMNPLL DQYLWHLQST |
| 451 | TTGNSLNQGT ATTTYGKITT GDFAYYRKNW LPGACIKQQK FSKNANQNYK |
| 501 | IPASGGDALL KYDTHTTLNG RWSNMAPGPP MATAGAGDSD FSNSQLIFAG |
| 551 | PNPSGNITTS SNNLLFTSEE EIATTNPRDT DMFGQIADNN QNATTAPHIA |
| 601 | NLDAMGIVPG MVWQNRDIYY QGPIWAKVPH TDGHFHPSPL MGGFGLKHPP |
| 651 | PQIFIKNTPV PANPNTTFSA ARINSFLIQY STGQVAVQID WEIQKEHSKR |
| 701 | WNPEVQFTSN YGTQNSMLWA PDNAGNYHEL RAIGSRFLTH HL |
| Example of wild-type AAVrh10 capsid protein | |
| (SEQ ID NO: 11) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPVEPSP |
| 151 | QRSPDSSTGI GKKGQQPAKK RLNFGQTGDS ESVPDPQPIG EPPAGPSGLG |
| 201 | SGTMAAGGGA PMADNNEGAD GVGSSSGNWH CDSTWLGDRV ITTSTRTWAL |
| 251 | PTYNNHLYKQ ISNGTSGGST NDNTYFGYST PWGYFDFNRF HCHFSPRDWQ |
| 301 | RLINNNWGFR PKRLNFKLEN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRTGN NFEFSYQFED VPFHSSYAHS QSLDRLMNPL IDQYLYYLSR |
| 451 | TQSTGGTAGT QQLLFSQAGP NNMSAQAKNW LPGPCYRQQR VSTTLSQNNN |
| 501 | SNFAWIGATK YHLNGRDSLV NPGVAMATHK DDEERFFPSS GVLMFGKQGA |
| 551 | GKDNVDYSSV MLISEEEIKI TNPVATEQYG VVADNLQQQN AAPIVGAVNS |
| 601 | QGALPGMVWQ NRDVYLQGPI WAKIPHTDGN FHPSPLMGGF GLKHPPPQIL |
| 651 | IKNTPVPADP PTTFSQAKLA SFITQYSTGQ VSVEIEWELQ KENSKRWNPE |
| 701 | IQYTSNYYKS TNVDFAVNTD GTYSEPRPIG TRYLTRNL |

Also provided herein are nucleic acids encoding capsid proteins. A nucleic acid may comprise a sequence that encodes a capsid protein disclosed here (e.g., a capsid protein comprising one or more amino acid substitutions). A sequence encoding a capsid protein disclosed herein can be determined by one of ordinary skill in the art by known methods. A nucleic acid encoding a capsid protein may comprise a promoter or other regulatory sequence operably linked to the coding sequence. A nucleic acid encoding a capsid protein may be in the form of a plasmid, an mRNA, or another nucleic acid capable of being used by enzymes or machinery of a host cell to produce a capsid protein. Nucleic acids encoding capsid proteins as provided herein can be used to make AAV particles that can be used for delivering a gene to a cell. Methods of making AAV particles are known in the art. For example, see Scientific Reports volume 9, Article number: 13601 (2019); Methods Mol Biol. 2012; 798: 267-284; and www.thermofisher.com/us/en/home/clinical/cell-gene-therapy/gene-therapy/aav-production-workflow.html. Example sequences of nucleic acids encoding capsid proteins are provided below.

Example of a nucleotide sequence encoding AAV1 capsid protein:
(SEQ ID NO: 25)
atggctgccgatggttatcttccagattggctcgaggacaacctctctgagggcattcgcgagtggtgggacttga
aacctggagccccgaagcccaaagccaaccagcaaaagcaggacgacggccggggtctggtgcttcctggctacaa
gtacctcggacccttcaacggactcgacaagggggagcccgtcaacgcggcggacgcagcggccctcgagcacgac
aaggcctacgaccagcagctcaaagcgggtgacaatccgtacctgcggtataaccacgccgacgccgagtttcagg
agcgtctgcaagaagatacgtcttttgggggcaacctcgggcgagcagtcttccaggccaagaagcgggttctcga
acctctcggtctggttgaggaaggcgctaagacggctcctggaaagaaacgtccggtagagcagtcgccacaagag
ccagactcctcctcgggcatcggcaagacaggccagcagcccgctaaaaagagactcaattttggtcagactggcg
actcagagtcagtccccgatccacaacctctcggagaacctccagcaacccccgctgctgtgggacctactacaat
ggcttcaggcggtggcgcaccaatggcagacaataacgaaggcgccgacggagtgggtaatgcctcaggaaattgg
cattgcgattccacatggctgggcgacagagtcatcaccaccagcacccgcacctgggccttgcccacctacaata
accacctctacaagcaaatctccagtgcttcaacgggggccagcaacgacaaccactacttcggctacagcacccc
ctgggggtattttgatttcaacagattccactgccacttttcaccacgtgactggcagcgactcatcaacaacaat
tggggattccggcccaagagactcaacttcaaactcttcaacatccaagtcaaggaggtcacgacgaatgatggcg
tcacaaccatcgctaataaccttaccagcacggttcaagtcttctcggactcggagtaccagcttccgtacgtcct
cggctctgcgcaccagggctgcctccctccgttcccggcggacgtgttcatgattccgcaatacggctacctgacg
ctcaacaatggcagccaagccgtgggacgttcatccttttactgcctggaatatttcccttctcagatgctgagaa
cgggcaacaactttaccttcagctacacctttgaggaagtgcctttccacagcagctacgcgcacagccagagcct
ggaccggctgatgaatcctctcatcgaccaatacctgtattacctgaacagaactcaaaatcagtccggaagtgcc
caaaacaaggacttgctgtttagccgtgggtctccagctggcatgtctgttcagcccaaaaactggctacctggac
cctgttatcggcagcagcgcgtttctaaaacaaaaacagacaacaacaacagcaattttacctggactggtgcttc
aaaatataacctcaatgggcgtgaatccatcatcaaccctggcactgctatggcctcacacaaagacgacgaagac
aagttctttcccatgagcggtgtcatgatttttggaaaagagagcgccggagcttcaaacactgcattggacaatg
tcatgattacagacgaagaggaaattaaagccactaaccctgtggccaccgaaagatttgggaccgtggcagtcaa
tttccagagcagcagcacagaccctgcgaccggagatgtgcatgctatgggagcattacctggcatggtgtggcaa
gatagagacgtgtacctgcagggtcccatttgggccaaaattcctcacacagatggacactttcacccgtctcctc
ttatgggcggctttggactcaagaacccgcctcctcagatcctcatcaaaaacacgcctgttcctgcgaatcctcc
ggcggagttttcagctacaaagtttgcttcattcatcacccaatactccacaggacaagtgagtgtggaaattgaa
tgggagctgcagaaagaaaacagcaagcgctggaatcccgaagtgcagtacacatccaattatgcaaaatctgcca
acgttgattttactgtggacaacaatggactttatactgagcctcgccccattggcacccgttaccttacccgtcc
cctgtaa
Example of a nucleotide sequence encoding AAV2 capsid protein:
(SEQ ID NO: 26)
atggctgccgatggttatcttccagattggctcgaggacactctctctgaaggaataagacagtggtggaagctca
aacctggcccaccaccaccaaagcccgcagagcggcataaggacgacagcaggggtcttgtgcttcctgggtacaa
gtacctcggacccttcaacggactcgacaagggagagccggtcaacgaggcagacgccgcggccctcgagcacgac
aaagcctacgaccggcagctcgacagcggagacaacccgtacctcaagtacaaccacgccgacgcggagtttcagg
agcgccttaaagaagatacgtcttttgggggcaacctcggacgagcagtcttccaggcgaaaaagagggttcttga
acctctgggcctggttgaggaacctgttaagacggctccgggaaaaaagaggccggtagagcactctcctgtggag
ccagactcctcctcgggaaccggaaaggcgggccagcagcctgcaagaaaaagattgaattttggtcagactggag
acgcagactcagtacctgacccccagcctctcggacagccaccagcagccccctctggtctgggaactaatacgat
ggctacaggcagtggcgcaccaatggcagacaataacgagggcgccgacggagtgggtaattcctccggaaattgg
cattgcgattccacatggatgggcgacagagtcatcaccaccagcacccgaacctgggccctgcccacctacaaca
accacctctacaaacaaatttccagccaatcaggagcctcgaacgacaatcactactttggctacagcaccccttg
ggggtattttgacttcaacagattccactgccacttttcaccacgtgactggcaaagactcatcaacaacaactgg
ggattccgacccaagagactcaacttcaagctctttaacattcaagtcaaagaggtcacgcagaatgacggtacga
cgacgattgccaataaccttaccagcacggttcaggtgtttactgactcggagtaccagctcccgtacgtcctcgg
ctcggcgcatcaaggatgcctcccgccgttcccagcagacgtcttcatggtgccacagtatggatacctcaccctg
aacaacgggagtcaggcagtaggacgctcttcattttactgcctggagtactttccttctcagatgctgcgtaccg
gaaacaactttaccttcagctacacttttgaggacgttcctttccacagcagctacgctcacagccagagtctgga
ccgtctcatgaatcctctcatcgaccagtacctgtattacttgagcagaacaaacactccaagtggaaccaccacg
cagtcaaggcttcagttttctcaggccggagcgagtgacattcgggaccagtctaggaactggcttcctggaccct
gttaccgccagcagcgagtatcaaagacatctgcggataacaacaacagtgaatactcgtggactggagctaccaa
gtaccacctcaatggcagagactctctggtgaatccgggcccggccatggcaagccacaaggacgatgaagaaaag
ttttttcctcagagcggggttctcatctttgggaagcaaggctcagagaaaacaaatgtggacattgaaaaggtca
tgattacagacgaagaggaaatcaggacaaccaatcccgtggctacggagcagtatggttctgtatctaccaacct
ccagagaggcaacagacaagcagctaccgcagatgtcaacacacaaggcgttcttccaggcatggtctggcaggac
agagatgtgtaccttcaggggcccatctgggcaaagattccacacacggacggacattttcacccctctcccctca
tgggtggattcggacttaaacaccctcctccacagattctcatcaagaacaccccggtacctgcgaatccttcgac
caccttcagtgcggcaaagtttgcttccttcatcacacagtactccacgggacaggtcagcgtggagatcgagtgg
gagctgcagaaggaaaacagcaaacgctggaatcccgaaattcagtacacttccaactacaacaagtctgttaatg
tggactttactgtggacactaatggcgtgtattcagagcctcgccccattggcaccagatacctgactcgtaatct
gtaa
Example of a nucleotide sequence encoding AAV3 capsid protein:
(SEQ ID NO: 27)
atggctgctgacggttatcttccagattggctcgaggacaacctttctgaaggcattcgtgagtggtgggctctga
aacctggagtccctcaacccaaagcgaaccaacaacaccaggacaaccgtcggggtcttgtgcttccgggttacaa
atacctcggacccggtaacggactcgacaaaggagagccggtcaacgaggcggacgcggcagccctcgaacacgac
aaagcttacgaccagcagctcaaggccggtgacaacccgtacctcaagtacaaccacgccgacgccgagtttcagg
agcgtcttcaagaagatacgtcttttgggggcaaccttggcagagcagtcttccaggccaaaaagaggatccttga
gcctcttggtctggttgaggaagcagctaaaacggctcctggaaagaagggggctgtagatcagtctcctcaggaa
ccggactcatcatctggtgttggcaaatcgggcaaacagcctgccagaaaaagactaaatttcggtcagactggag
actcagagtcagtcccagaccctcaacctctcggagaaccaccagcagcccccacaagtttgggatctaatacaat
ggcttcaggcggtggcgcaccaatggcagacaataacgagggtgccgatggagtgggtaattcctcaggaaattgg
cattgcgattcccaatggctgggcgacagagtcatcaccaccagcaccagaacctgggccctgcccacttacaaca
accatctctacaagcaaatctccagccaatcaggagcttcaaacgacaaccactactttggctacagcaccccttg
ggggtattttgactttaacagattccactgccacttctcaccacgtgactggcagcgactcattaacaacaactgg
ggattccggcccaagaaactcagcttcaagctcttcaacatccaagttagaggggtcacgcagaacgatggcacga
cgactattgccaataaccttaccagcacggttcaagtgtttacggactcggagtatcagctcccgtacgtgctcgg
gtcggcgcaccaaggctgtctcccgccgtttccagcggacgtcttcatggtccctcagtatggatacctcaccctg
aacaacggaagtcaagcggtgggacgctcatccttttactgcctggagtacttcccttcgcagatgctaaggactg
gaaataacttccaattcagctataccttcgaggatgtaccttttcacagcagctacgctcacagccagagtttgga
tcgcttgatgaatcctcttattgatcagtatctgtactacctgaacagaacgcaaggaacaacctctggaacaacc
aaccaatcacggctgctttttagccaggctgggcctcagtctatgtctttgcaggccagaaattggctacctgggc
cctgctaccggcaacagagactttcaaagactgctaacgacaacaacaacagtaactttccttggacagcggccag
caaatatcatctcaatggccgcgactcgctggtgaatccaggaccagctatggccagtcacaaggacgatgaagaa
aaatttttccctatgcacggcaatctaatatttggcaaagaagggacaacggcaagtaacgcagaattagataatg
taatgattacggatgaagaagagattcgtaccaccaatcctgtggcaacagagcagtatggaactgtggcaaataa
cttgcagagctcaaatacagctcccacgactggaactgtcaatcatcagggggccttacctggcatggtgtggcaa
gatcgtgacgtgtaccttcaaggacctatctgggcaaagattcctcacacggatggacactttcatccttctcctc
tgatgggaggctttggactgaaacatccgcctcctcaaatcatgatcaaaaatactccggtaccggcaaatcctcc
gacgactttcagcccggccaagtttgcttcatttatcactcagtactccactggacaggtcagcgtggaaattgag
tgggagctacagaaagaaaacagcaaacgttggaatccagagattcagtacacttccaactacaacaagtctgtta
atgtggactttactgtagacactaatggtgtttatagtgaacctcgccctattggaacccggtatctcacacgaaa
cttgtga
Example of a nucleotide sequence encoding AAV4 capsid protein:
(SEQ ID NO: 28)
atgactgacggttaccttccagattggctagaggacaacctctctgaaggcgttcgagagtggtgggcgctgcaac
ctggagcccctaaacccaaggcaaatcaacaacatcaggacaacgctcggggtcttgtgcttccgggttacaaata
cctcggacccggcaacggactcgacaagggggaacccgtcaacgcagcggacgcggcagccctcgagcacgacaag
gcctacgaccagcagctcaaggccggtgacaacccctacctcaagtacaaccacgccgacgcggagttccagcagc
ggcttcagggcgacacatcgtttgggggcaacctcggcagagcagtcttccaggccaaaaagagggttcttgaacc
tcttggtctggttgagcaagcgggtgagacggctcctggaaagaagagaccgttgattgaatccccccagcagccc
gactcctccacgggtatcggcaaaaaaggcaagcagccggctaaaaagaagctcgttttcgaagacgaaactggag
caggcgacggaccccctgagggatcaacttccggagccatgtctgatgacagtgagatgcgtgcagcagctggcgg
agctgcagtcgagggcggacaaggtgccgatggagtgggtaatgcctcgggtgattggcattgcgattccacctgg
tctgagggccacgtcacgaccaccagcaccagaacctgggtcttgcccacctacaacaaccacctctacaagcgac
tcggagagagcctgcagtccaacacctacaacggattctccaccccctggggatactttgacttcaaccgcttcca
ctgccacttctcaccacgtgactggcagcgactcatcaacaacaactggggcatgcgacccaaagccatgcgggtc
aaaatcttcaacatccaggtcaaggaggtcacgacgtcgaacggcgagacaacggtggctaataaccttaccagca
cggttcagatctttgcggactcgtcgtacgaactgccgtacgtgatggatgcgggtcaagagggcagcctgcctcc
ttttcccaacgacgtctttatggtgccccagtacggctactgtggactggtgaccggcaacacttcgcagcaacag
actgacagaaatgccttctactgcctggagtactttccttcgcagatgctgcggactggcaacaactttgaaatta
cgtacagttttgagaaggtgcctttccactcgatgtacgcgcacagccagagcctggaccggctgatgaaccctct
catcgaccagtacctgtggggactgcaatcgaccaccaccggaaccaccctgaatgccgggactgccaccaccaac
tttaccaagctgcggcctaccaacttttccaactttaaaaagaactggctgcccgggccttcaatcaagcagcagg
gcttctcaaagactgccaatcaaaactacaagatccctgccaccgggtcagacagtctcatcaaatacgagacgca
cagcactctggacggaagatggagtgccctgacccccggacctccaatggccacggctggacctgcggacagcaag
ttcagcaacagccagctcatctttgcggggcctaaacagaacggcaacacggccaccgtacccgggactctgatct
tcacctctgaggaggagctggcagccaccaacgccaccgatacggacatgtggggcaacctacctggcggtgacca
gagcaacagcaacctgccgaccgtggacagactgacagccttgggagccgtgcctggaatggtctggcaaaacaga
gacatttactaccagggtcccatttgggccaagattcctcataccgatggacactttcacccctcaccgctgattg
gtgggtttgggctgaaacacccgcctcctcaaatttttatcaagaacaccccggtacctgcgaatcctgcaacgac
cttcagctctactccggtaaactccttcattactcagtacagcactggccaggtgtcggtgcagattgactgggag
atccagaaggagcggtccaaacgctggaaccccgaggtccagtttacctccaactacggacagcaaaactctctgt
tgtgggctcccgatgcggctgggaaatacactgagcctagggctatcggtacccgctacctcacccaccacctgta
ataacctgttaatcaataaaccggtttattcgtttcagttgaactttggtctccgtgtccttcttatcttatctcg
tttcc
Example of a nucleotide sequence encoding AAV5 capsid protein:
(SEQ ID NO: 29)
atgtcttttgttgatcaccctccagattggttggaagaagttggtgaaggtcttcgcgagtttttgggccttgaag
cgggcccaccgaaaccaaaacccaatcagcagcatcaagatcaagcccgtggtcttgtgctgcctggttataacta
tctcggacccggaaacggtctcgatcgaggagagcctgtcaacagggcagacgaggtcgcgcgagagcacgacatc
tcgtacaacgagcagcttgaggcgggagacaacccctacctcaagtacaaccacgcggacgccgagtttcaggaga
agctcgccgacgacacatccttcgggggaaacctcggaaaggcagtctttcaggccaagaaaagggttctcgaacc
ttttggcctggttgaagagggtgctaagacggcccctaccggaaagcggatagacgaccactttccaaaaagaaag
aaggctcggaccgaagaggactccaagccttccacctcgtcagacgccgaagctggacccagcggatcccagcagc
tgcaaatcccagcccaaccagcctcaagtttgggagctgatacaatgtctgcgggaggtggcggcccattgggcga
caataaccaaggtgccgatggagtgggcaatgcctcgggagattggcattgcgattccacgtggatgggggacaga
gtcgtcaccaagtccacccgaacctgggtgctgcccagctacaacaaccaccagtaccgagagatcaaaagcggct
ccgtcgacggaagcaacgccaacgcctactttggatacagcaccccctgggggtactttgactttaaccgcttcca
cagccactggagcccccgagactggcaaagactcatcaacaactactggggcttcagaccccggtccctcagagtc
aaaatcttcaacattcaagtcaaagaggtcacggtgcaggactccaccaccaccatcgccaacaacctcacctcca
ccgtccaagtgtttacggacgacgactaccagctgccctacgtcgtcggcaacgggaccgagggatgcctgccggc
cttccctccgcaggtctttacgctgccgcagtacggttacgcgacgctgaaccgcgacaacacagaaaatcccacc
gagaggagcagcttcttctgcctagagtactttcccagcaagatgctgagaacgggcaacaactttgagtttacct
acaactttgaggaggtgcccttccactccagcttcgctcccagtcagaacctgttcaagctggccaacccgctggt
ggaccagtacttgtaccgcttcgtgagcacaaataacactggcggagtccagttcaacaagaacctggccgggaga
tacgccaacacctacaaaaactggttcccggggcccatgggccgaacccagggctggaacctgggctccggggtca
accgcgccagtgtcagcgccttcgccacgaccaataggatggagctcgagggcgcgagttaccaggtgcccccgca
gccgaacggcatgaccaacaacctccagggcagcaacacctatgccctggagaacactatgatcttcaacagccag
ccggcgaacccgggcaccaccgccacgtacctcgagggcaacatgctcatcaccagcgagagcgagacgcagccgg
tgaaccgcgtggcgtacaacgtcggcgggcagatggccaccaacaaccagagctccaccactgcccccgcgaccgg
cacgtacaacctccaggaaatcgtgcccggcagcgtgtggatggagagggacgtgtacctccaaggacccatctgg
gccaagatcccagagacgggggcgcactttcacccctctccggccatgggcggattcggactcaaacacccaccgc
ccatgatgctcatcaagaacacgcctgtgcccggaaatatcaccagcttctcggacgtgcccgtcagcagcttcat
cacccagtacagcaccgggcaggtcaccgtggagatggagtgggagctcaagaaggaaaactccaagaggtggaac
ccagagatccagtacacaaacaactacaacgacccccagtttgtggactttgccccggacagcaccggggaataca
gaaccaccagacctatcggaacccgataccttacccgacccctttaa
Example of a nucleotide sequence encoding AAV6 capsid protein:
(SEQ ID NO: 30)
atggctgccgatggttatcttccagattggctcgaggacaacctctctgagggcattcgcgagtggtgggacttga
aacctggagccccgaaacccaaagccaaccagcaaaagcaggacgacggccggggtctggtgcttcctggctacaa
gtacctcggacccttcaacggactcgacaagggggagcccgtcaacgcggcggatgcagcggccctcgagcacgac
aaggcctacgaccagcagctcaaagcgggtgacaatccgtacctgcggtataaccacgccgacgccgagtttcagg
agcgtctgcaagaagatacgtcttttgggggcaacctcgggcgagcagtcttccaggccaagaagagggttctcga
accttttggtctggttgaggaaggtgctaagacggctcctggaaagaaacgtccggtagagcagtcgccacaagag
ccagactcctcctcgggcattggcaagacaggccagcagcccgctaaaaagagactcaattttggtcagactggcg
actcagagtcagtccccgacccacaacctctcggagaacctccagcaacccccgctgctgtgggacctactacaat
ggcttcaggcggtggcgcaccaatggcagacaataacgaaggcgccgacggagtgggtaatgcctcaggaaattgg
cattgcgattccacatggctgggcgacagagtcatcaccaccagcacccgaacatgggccttgcccacctataaca
accacctctacaagcaaatctccagtgcttcaacgggggccagcaacgacaaccactacttcggctacagcacccc
ctgggggtattttgatttcaacagattccactgccatttctcaccacgtgactggcagcgactcatcaacaacaat
tggggattccggcccaagagactcaacttcaagctcttcaacatccaagtcaaggaggtcacgacgaatgatggcg
tcacgaccatcgctaataaccttaccagcacggttcaagtcttctcggactcggagtaccagttgccgtacgtcct
cggctctgcgcaccagggctgcctccctccgttcccggcggacgtgttcatgattccgcagtacggctacctaacg
ctcaacaatggcagccaggcagtgggacggtcatccttttactgcctggaatatttcccatcgcagatgctgagaa
cgggcaataactttaccttcagctacaccttcgaggacgtgcctttccacagcagctacgcgcacagccagagcct
ggaccggctgatgaatcctctcatcgaccagtacctgtattacctgaacagaactcagaatcagtccggaagtgcc
caaaacaaggacttgctgtttagccgggggtctccagctggcatgtctgttcagcccaaaaactggctacctggac
cctgttaccggcagcagcgcgtttctaaaacaaaaacagacaacaacaacagcaactttacctggactggtgcttc
aaaatataaccttaatgggcgtgaatctataatcaaccctggcactgctatggcctcacacaaagacgacaaagac
aagttctttcccatgagcggtgtcatgatttttggaaaggagagcgccggagcttcaaacactgcattggacaatg
tcatgatcacagacgaagaggaaatcaaagccactaaccccgtggccaccgaaagatttgggactgtggcagtcaa
tctccagagcagcagcacagaccctgcgaccggagatgtgcatgttatgggagccttacctggaatggtgtggcaa
gacagagacgtatacctgcagggtcctatttgggccaaaattcctcacacggatggacactttcacccgtctcctc
tcatgggggctttggacttaagcacccgcctcctcagatcctcatcaaaaacacgcctgttcctgcgaatcctcc
ggcagagttttcggctacaaagtttgcttcattcatcacccagtattccacaggacaagtgagcgtggagattgaa
tgggagctgcagaaagaaaacagcaaacgctggaatcccgaagtgcagtatacatctaactatgcaaaatctgcca
acgttgatttcactgtggacaacaatggactttatactgagcctcgccccattggcacccgttacctcacccgtcc
cctgtaat
Example of a nucleotide sequence encoding AAV7 capsid protein:
(SEQ ID NO: 31)
atggctgccgatggttatcttccagattggctcgaggacaacctctctgagggcattcgcgagtggtgggacctga
aacctggagccccgaaacccaaagccaaccagcaaaagcaggacaacggccggggtctggtgcttcctggctacaa
gtacctcggacccttcaacggactcgacaagggggagcccgtcaacgcggcggacgcagcggccctcgagcacgac
aaggcctacgaccagcagctcaaagcgggtgacaatccgtacctgcggtataaccacgccgacgccgagtttcagg
agcgtctgcaagaagatacgtcatttgggggcaacctcgggcgagcagtcttccaggccaagaagcgggttctcga
acctctcggtctggttgaggaaggcgctaagacggctcctgcaaagaagagaccggtagagccgtcacctcagcgt
tcccccgactcctccacgggcatcggcaagaaaggccagcagcccgccagaaagagactcaatttcggtcagactg
gcgactcagagtcagtccccgaccctcaacctctcggagaacctccagcagcgccctctagtgtgggatctggtac
agtggctgcaggcggtggcgcaccaatggcagacaataacgaaggtgccgacggagtgggtaatgcctcaggaaat
tggcattgcgattccacatggctgggcgacagagtcattaccaccagcacccgaacctgggccctgcccacctaca
acaaccacctctacaagcaaatctccagtgaaactgcaggtagtaccaacgacaacacctacttcggctacagcac
cccctgggggtattttgactttaacagattccactgccacttctcaccacgtgactggcagcgactcatcaacaac
aactggggattccggcccaagaagctgcggttcaagctcttcaacatccaggtcaaggaggtcacgacgaatgacg
gcgttacgaccatcgctaataaccttaccagcacgattcaggtattctcggactcggaataccagctgccgtacgt
cctcggctctgcgcaccagggctgcctgcctccgttcccggcggacgtcttcatgattcctcagtacggctacctg
actctcaacaatggcagtcagtctgtgggacgttcctccttctactgcctggagtacttcccctctcagatgctga
gaacgggcaacaactttgagttcagctacagcttcgaggacgtgcctttccacagcagctacgcacacagccagag
cctggaccggctgatgaatcccctcatcgaccagtacttgtactacctggccagaacacagagtaacccaggaggc
acagctggcaatcgggaactgcagttttaccaggggggccttcaactatggccgaacaagccaagaattggttac
ctggaccttgcttccggcaacaaagagtctccaaaacgctggatcaaaacaacaacagcaactttgcttggactgg
tgccaccaaatatcacctgaacggcagaaactcgttggttaatcccggcgtcgccatggcaactcacaaggacgac
gaggaccgctttttcccatccagcggagtcctgatttttggaaaaactggagcaactaacaaaactacattggaaa
atgtgttaatgacaaatgaagaagaaattcgtcctactaatcctgtagccacggaagaatacgggatagtcagcag
caacttacaagcggctaatactgcagcccagacacaagttgtcaacaaccagggagccttacctggcatggtctgg
cagaaccgggacgtgtacctgcagggtcccatctgggccaagattcctcacacggatggcaactttcacccgtctc
ctttgatgggcggctttggacttaaacatccgcctcctcagatcctgatcaagaacactcccgttcccgctaatcc
tccggaggtgtttactcctgccaagtttgcttcgttcatcacacagtacagcaccggacaagtcagcgtggaaatc
gagtgggagctgcagaaggaaaacagcaagcgctggaacccggagattcagtacacctccaactttgaaaagcaga
ctggtgtggactttgccgttgacagccagggtgtttactctgagcctcgccctattggcactcgttacctcacccg
taatctgtaa
Example of a nucleotide sequence encoding AAV8 capsid protein:
(SEQ ID NO: 32)
atggctgccgatggttatcttccagattggctcgaggacaacctctctgagggcattcgcgagtggtgggcgctga
aacctggagccccgaagcccaaagccaaccagcaaaagcaggacgacggccggggtctggtgcttcctggctacaa
gtacctcggacccttcaacggactcgacaagggggagcccgtcaacgcggcggacgcagcggccctggagcacgac
aaggcctacgaccagcagctgcaggcgggtgacaatccgtacctgcggtataaccacgccgacgccgagtttcagg
agcgtctgcaagaagatacgtcttttgggggcaacctcgggcgagcagtcttccaggccaagaagcgggttctcga
acctctcggtctggttgaggaaggcgctaagacggctcctggaaagaagagaccggtagagccatcaccccagcgt
tctccagactcctctacgggcatcggcaagaaaggccaacagcccgccagaaaaagactcaattttggtcagactg
gcgactcagagtcagttccagaccctcaacctctcggagaacctccagcagcgccctctggtgtgggacctaatac
aatggctgcaggcggtggcgcaccaatggcagacaataacgaaggcgccgacggagtgggtagttcctcgggaaat
tggcattgcgattccacatggctgggcgacagagtcatcaccaccagcacccgaacctgggccctgcccacctaca
acaaccacctctacaagcaaatctccaacgggacatcgggaggagccaccaacgacaacacctacttcggctacag
caccccctgggggtattttgactttaacagattccactgccacttttcaccacgtgactggcagcgactcatcaac
aacaactggggattccggcccaagagactcagcttcaagctcttcaacatccaggtcaaggaggtcacgcagaatg
aaggcaccaagaccatcgccaataacctcaccagcaccatccaggtgtttacggactcggagtaccagctgccgta
cgttctcggctctgcccaccagggctgcctgcctccgttcccggcggacgtgttcatgattccccagtacggctac
ctaacactcaacaacggtagtcaggccgtgggacgctcctccttctactgcctggaatactttccttcgcagatgc
tgagaaccggcaacaacttccagtttacttacaccttcgaggacgtgcctttccacagcagctacgcccacagcca
gagcttggaccggctgatgaatcctctgattgaccagtacctgtactacttgtctcggactcaaacaacaggaggc
acggcaaatacgcagactctgggcttcagccaaggtgggcctaatacaatggccaatcaggcaaagaactggctgc
caggaccctgttaccgccaacaacgcgtctcaacgacaaccgggcaaaacaacaatagcaactttgcctggactgc
tgggaccaaataccatctgaatggaagaaattcattggctaatcctggcatcgctatggcaacacacaaagacgac
gaggagcgtttttttcccagtaacgggatcctgatttttggcaaacaaaatgctgccagagacaatgcggattaca
gcgatgtcatgctcaccagcgaggaagaaatcaaaaccactaaccctgtggctacagaggaatacggtatcgtggc
agataacttgcagcagcaaaacacggctcctcaaattggaactgtcaacagccagggggccttacccggtatggtc
tggcagaaccgggacgtgtacctgcagggtcccatctgggccaagattcctcacacggacggcaacttccacccgt
ctccgctgatgggcggctttggcctgaaacatcctccgcctcagatcctgatcaagaacacgcctgtacctgcgga
tcctccgaccaccttcaaccagtcaaagctgaactctttcatcacgcaatacagcaccggacaggtcagcgtggaa
attgaatgggagctgcagaaggaaaacagcaagcgctggaaccccgagatccagtacacctccaactactacaaat
ctacaagtgtggactttgctgttaatacagaaggcgtgtactctgaaccccgccccattggcacccgttacctcac
ccgtaatctgtaa
Example of a nucleotide sequence encoding AAV9 capsid protein:
(SEQ ID NO: 33)
atggctgccgatggttatcttccagattggctcgaggacaaccttagtgaaggaattcgcgagtggtgggctttga
aacctggagcccctcaacccaaggcaaatcaacaacatcaagacaacgctcgaggtcttgtgcttccgggttacaa
ataccttggacccggcaacggactcgacaagggggagccggtcaacgcagcagacgcggcggccctcgagcacgac
aaggcctacgaccagcagctcaaggccggagacaacccgtacctcaagtacaaccacgccgacgccgagttccagg
agcggctcaaagaagatacgtcttttgggggcaacctcgggcgagcagtcttccaggccaaaaagaggcttcttga
acctcttggtctggttgaggaagcggctaagacggctcctggaaagaagaggcctgtagagcagtctcctcaggaa
ccggactcctccgcgggtattggcaaatcgggtgcacagcccgctaaaaagagactcaatttcggtcagactggcg
acacagagtcagtcccagaccctcaaccaatcggagaacctcccgcagccccctcaggtgtgggatctottacaat
ggcttcaggtggtggcgcaccagtggcagacaataacgaaggtgccgatggagtgggtagttcctcgggaaattgg
cattgcgattcccaatggctgggggacagagtcatcaccaccagcacccgaacctgggccctgcccacctacaaca
atcacctctacaagcaaatctccaacagcacatctggaggatottcaaatgacaacgcctacttcggctacagcac
cccctgggggtattttgacttcaacagattccactgccacttctcaccacgtgactggcagcgactcatcaacaac
aactggggattccggcctaagcgactcaacttcaagctcttcaacattcaggtcaaagaggttacggacaacaatg
gagtcaagaccatcgccaataaccttaccagcacggtccaggtcttcacggactcagactatcagctcccgtacgt
gctcgggtcggctcacgagggctgcctcccgccgttcccagcggacgttttcatgattcctcagtacgggtatctg
acgcttaatgatggaagccaggccgtgggtcgttcgtccttttactgcctggaatatttcccgtcgcaaatgctaa
gaacgggtaacaacttccagttcagctacgagtttgagaacgtacctttccatagcagctacgctcacagccaaag
cctggaccgactaatgaatccactcatcgaccaatacttgtactatctctcaaagactattaacggttctggacag
aatcaacaaacgctaaaattcagtgtggccggacccagcaacatggctgtccagggaagaaactacatacctggac
ccagctaccgacaacaacgtgtctcaaccactgtgactcaaaacaacaacagcgaatttgcttggcctggagcttc
ttcttgggctctcaatggacgtaatagcttgatgaatcctggacctgctatggccagccacaaagaaggagaggac
cgtttctttcctttgtctggatctttaatttttggcaaacaaggaactggaagagacaacgtggatgcggacaaag
tcatgataaccaacgaagaagaaattaaaactactaacccggtagcaacggagtcctatggacaagtggccacaaa
ccaccagagtgcccaagcacaggcgcagaccggctgggttcaaaaccaaggaatacttccgggtatggtttggcag
gacagagatgtgtacctgcaaggacccatttgggccaaaattcctcacacggacggcaactttcacccttctccgc
tgatgggagggtttggaatgaagcacccgcctcctcagatcctcatcaaaaacacacctgtacctgcggatcctcc
aacggccttcaacaaggacaagctgaactctttcatcacccagtattctactggccaagtcagcgtggagatcgag
tgggagctgcagaaggaaaacagcaagcgctggaacccggagatccagtacacttccaactattacaagtctaata
atgttgaatttgctgttaatactgaaggtgtatatagtgaaccccgccccattggcaccagatacctgactcgtaa
tctgtaa
Example of a nucleotide sequence encoding AAV10 capsid protein:
(SEQ ID NO: 34)
atggctgccgatggttatcttccagattggctcgaggacaacctctctgagggcattcgcgagtggtgggacttga
aacctggagccccgaaacccaaagccaaccagcaaaagcaggacgacggccggggtctggtgcttcctggctacaa
gtacctcggacccttcaacggactcgacaagggggagcccgtcaacgcggcggacgcagcggccctcgagcacgac
aaggcctacgaccagcagctcaaagcgggtgacaatccgtacctgcggtataaccacgccgacgccgagtttcagg
agcgtctgcaagaagatacgtcttttgggggcaacctcgggcgagcagtcttccaggccaagaagcgggttctcga
acctctcggtctggttgaggaaggcgctaagacggctcctggaaagaagagaccggtagagccatcaccccagcgt
tctccagactcctctacgggcatcggcaagaaaggccagcagcccgcgaaaaagagactcaactttgggcagactg
gcgactcagagtcagtgcccgaccctcaaccaatcggagaaccccccgcaggcccctctggtctgggatctggtac
aatggctgcaggcggtggcgctccaatggcagacaataacgaaggcgccgacggagtgggtagttcctcaggaaat
tggcattgcgattccacatggctgggcgacagagtcatcaccaccagcacccgaacctgggccctccccacctaca
acaaccacctctacaagcaaatctccaacgggacttcgggaggaagcaccaacgacaacacctacttcggctacag
caccccctgggggtattttgactttaacagattccactgccacttctcaccacgtgactggcagcgactcatcaac
aacaactggggattccggcccaagagactcaacttcaagctcttcaacatccaggtcaaggaggtcacgcagaatg
aaggcaccaagaccatcgccaataaccttaccagcacgattcaggtctttacggactcggaataccagctcccgta
cgtcctcggctctgcgcaccagggctgcctgcctccgttcccggcggacgtcttcatgattcctcagtacgggtac
ctgactctgaacaatggcagtcaggccgtgggccgttcctccttctactgcctggagtactttccttctcaaatgc
tgagaacgggcaacaactttgagttcagctaccagtttgaggacgtgccttttcacagcagctacgcgcacagcca
aagcctggaccggctgatgaaccccctcatcgaccagtacctgtactacctgtctcggactcagtccacgggaggt
accgcaggaactcagcagttgctattttctcaggccgggcctaataacatgtcggctcaggccaaaaactggctac
ccgggccctgctaccggcagcaacgcgtctccacgacactgtcgcaaaataacaacagcaactttgcctggaccgg
tgccaccaagtatcatctgaatggcagagactctctggtaaatcccggtgtcgctatggcaacccacaaggacgac
gaagagcgattttttccgtccagcggagtcttaatgtttgggaaacagggagctggaaaagacaacgtggactata
gcagcgttatgctaaccagtgaggaagaaattaaaaccaccaacccagtggccacagaacagtacggcgtggtggc
cgataacctgcaacagcaaaacgccgctcctattgtaggggccgtcaacagtcaaggagccttacctggcatggtc
tggcagaaccgggacgtgtacctgcagggtcctatctgggccaagattcctcacacggacggaaactttcatccct
cgccgctgatgggaggctttggactgaaacacccgcctcctcagatcctgattaagaatacacctgttcccgcgga
tcctccaactaccttcagtcaagctaagctggcgtcgttcatcacgcagtacagcaccggacaggtcagcgtggaa
attgaatgggagctgcagaaagaaaacagcaaacgctggaacccagagattcaatacacttccaactactacaaat
ctacaaatgtggactttgctgttaacacagatggcacttattctgagcctcgccccatcggcacccgttacctcac
ccgtaatctgtaa

### Nucleic Acid Vectors

According to some aspects, provided herein are nucleic acid vectors that may be encapsidated by wild-type AAV capsids or any one of the AAV capsids (e.g., a capsid protein comprising one or more amino acid substitutions) as provided herein. In some embodiments, a nucleic acid vector as provided herein comprises a first inverted terminal repeat (ITR) and a second ITR. In some embodiments, the first ITR is modified. In some embodiments, the second ITR is modified. In some embodiments, a modification of an ITR comprises substitution of the entire D-sequence or substitution of part of a D-sequence. In some embodiments, a modification of an ITR comprises deletion of an entire D-sequence (e.g., the D-sequence of the left ITR or the right ITR) or deletion of part of a D-sequence (e.g., the distal 10 nucleotides of the ITR, relative to the terminus of the nucleic acid vector). For example, a modification of an ITR may in some embodiments comprise deletion or substitution of 1-20 nucleotides of the D-sequence. In some embodiments, the distal 1, 2, 3, 4, 5, 6, 7, 8, 9, 10, 11, 12, 13, 14, or 15 nucleotides of the D-sequence, relative to the terminus of the nucleic acid vector, are deleted or substituted. In some embodiments, the distal 10 nucleotides of the D-sequence, relative to the terminus of the nucleic acid vector, are deleted or substituted. In some embodiments, 1, 2, 3, 4, 5, 6, 7, 8, 9, 10, 11, 12, 13, 14, or 15 nucleotides in the middle of the D-sequence are deleted or substituted (e.g., 1, 2, 3, 4, 5, 6, 7, 8, 9, 10, 11, 12, 13, 14, or 15 contiguous nucleotides beginning 1, 2, 3, 4, 5, 6, 7, 8, 9, or 10 nucleotides from the 3′ or 5′ end of the D-sequence). In some embodiments, the proximal 1, 2, 3, 4, 5, 6, 7, 8, 9, 10, 11, 12, 13, 14, or 15 nucleotides of the D-sequence, relative to the terminus of the nucleic acid vector, are deleted or substituted. In some embodiments, the proximal 10 nucleotides of the D-sequence, relative to the terminus of the nucleic acid vector, are deleted or substituted. In some embodiments, a D-sequence comprises the sequence provided in SEQ ID NO: 16. In some embodiments, a D-sequence is defined by the sequence provided in SEQ ID NO: 16. In embodiments in which a portion or the entirety of a D-sequence of an ITR (e.g., the D-sequence of the left ITR or of the right ITR of a nucleic acid vector described herein) is substituted, the substituted sequence may be any alternative sequence described herein, such as an S-sequence or a GRE.

A nucleic acid vector may comprise one or more heterologous nucleic acid sequences encoding a gene of interest (e.g., a protein or polypeptide of interest) and one or more sequences comprising inverted terminal repeat (ITR) sequences (e.g., wild-type ITR sequences or modified ITR sequences) flanking the one or more heterologous nucleic acid sequences. In some embodiments, a nucleic acid vector is encapsidated within an AAV capsid forming an AAV particle. In some embodiments, a nucleic acid vector disclosed herein is encapsidated by a wild-type AAVrh74 capsid or another AAV capsid disclosed herein, such as an AAV capsid comprising one or more amino acid substitutions.

In some embodiments, a nucleic acid vector comprises native AAV genes or native AAV nucleotide sequences. In some embodiments, one or more native AAV genes or native AAV nucleotide sequences may be removed from a nucleic acid vector. In some embodiments, one or more native AAV genes or native AAV nucleotide sequences may be removed from a nucleic acid vector and replaced with a gene or interest.

A nucleic acid vector can be of any AAV serotype, such as AAV1, AAV2, AAV3, AAV4, AAV5, AAV6, AAV7, AAV8, AAV9, AAV10, AAV11, AAV12, AAV13, AAVrh10, or AAVrh74, or a combination of serotypes. In some embodiments, a nucleic acid vector encapsidated within an AAV capsid forms a pseudotyped AAV particle, such that the nucleic acid vector is of a serotype distinct from the AAV capsid in which it is encapsidated. For example, a nucleic acid vector of serotype AAV2 may be encapsidated within a capsid of serotype AAVrh74.

In some embodiments, a nucleic acid vector is single-stranded and comprises a first inverted terminal repeat (ITR) and a second ITR. As disclosed herein, the first ITR refers to the ITR at the 5′ terminus of the nucleic acid vector, and the second ITR refers to the ITR at the 3′ terminus of the nucleic acid vector. Each ITR in its native or wild-type form is or is about 145 nucleotides in length (e.g., about 140 nucleotides, about 145 nucleotides, about 150 nucleotides, about 155 nucleotides, about 160 nucleotides, or about 165 nucleotides) and comprises a D-sequence. Each ITR can independently be of any AAV serotype (e.g., AAV1, AAV2, AAV3, AAV4, AAV5, AAV6, AAV7, AAV8, AAV9, AAV10, AAV11, AAV12, AAV13, AAVrh10, or AAVrh74), or both ITRs may be of the same serotype. ITRs are described, for example, in Grimm et al. J. Virol. 80(1):426-439 (2006). Exemplary left ITR sequences are provided below. A right ITR has a nucleotide sequence which is the reverse complement of the corresponding left ITR (e.g., the AAV2 right ITR has a nucleotide sequence which is the reverse complement of the AAV2 left ITR).

Example of wild-type AAV1 left ITR:
(SEQ ID NO: 35)
TTGCCCACTCCCTCTCTGCGCGCTCGCTCGCTCGGTGGGGCCTGCGGAC
CAAAGGTCCGCAGACGGCAGAGGTCTCCTCTGCCGGCCCCACCGAGCGA
GCGAGCGCGCAGAGAGGGAGTGGGCAACTCCATCACTAGGGGTAA
Example of wild-type AAV2 left ITR:
(SEQ ID NO: 12)
TTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGA
GCGAGCGCGCAGAGAGGGAGTGGCCAACTCCATCACTAGGGGTTCCT
Example of wild-type AAV3 left ITR:
(SEQ ID NO: 13)
TTGGCCACTCCCTCTATGCGCACTCGCTCGCTCGGTGGGGCCTGGCGAC
CAAAGGTCGCCAGACGGACGTGCTTTGCACGTCCGGCCCCACCGAGCGA
GCGAGTGCGCATAGAGGGAGTGGCCAACTCCATCACTAGAGGTATGGC
Example of wild-type AAV4 left ITR:
(SEQ ID NO: 36)
TTGGCCACTCCCTCTATGCGCGCTCGCTCACTCACTCGGCCCTGGAGAC
CAAAGGTCTCCAGACTGCCGGCCTCTGGCCGGCAGGGCCGAGTGAGTGA
GCGAGCGCGCATAGAGGGAGTGGCCAACTCCATCATCTAGGTTTGCCC
Example of wild-type AAV5 left ITR:
(SEQ ID NO: 14)
CTCTCCCCCCTGTCGCGTTCGCTCGCTCGCTGGCTCGTTTGGGGGGGTG
GCAGCTCAAAGAGCTGCCAGACGACGGCCCTCTGGCCGTCGCCCCCCCA
AACGAGCCAGCGAGCGAGCGAACGCGACAGGGGGGAGAGTGCCACACTC
TCAAGCAAGGGGGTTTTGTA
Example of wild-type AAV6 left ITR:
(SEQ ID NO: 15)
TTGCCCACTCCCTCTATGCGCGCTCGCTCGCTCGGTGGGGCCTGCGGAC
CAAAGGTCCGCAGACGGCAGAGCTCTGCTCTGCCGGCCCCACCGAGCGA
GCGAGCGCGCATAGAGGGAGTGGGCAACTCCATCACTAGGGGTA

In some embodiments, a nucleic acid vector comprises a modification (e.g., a deletion or a substitution) of a D-sequence of an ITR. In some embodiments, a nucleic acid vector comprises a modification (e.g., a deletion or a substitution) of a D-sequence of a left ITR. In some embodiments, a nucleic acid vector comprises a modification (e.g., a deletion or a substitution) of a D-sequence of a right ITR. In some embodiments, a nucleic acid vector comprises a modification (e.g., a deletion or a substitution) of a D-sequence of both a left ITR and a right ITR. In some embodiments, a nucleic acid vector comprises a modification (e.g., a deletion or a substitution) of either a left ITR or a right ITR, but not both (i.e., the nucleic acid vector comprises a modification of only one ITR).

The ITR sequence comprises a terminal sequence at the 5′ or 3′ end of the AAV genome which forms a palindromic double-stranded T-shaped hairpin structure, and an additional sequence which remains single-stranded (i.e., is not part of the T-shaped hairpin structure), termed the D-sequence. The D-sequence of an ITR is typically approximately 20 (e.g., about 15, 16, 17, 18, 19, 20, 21, 22, 23, 24, or 25) nucleotides located at the distal (relative to the terminus of the nucleic acid vector) end of the ITR (i.e., the 3′ end of the left ITR or the 5′ end of the right ITR), and corresponds to the sequence of CTCCATCACTAGGGGTTCCT (SEQ ID NO: 16) of the wild-type AAV2 left ITR of SEQ ID NO: 12. The D-sequence of an ITR in some embodiments comprises, consists essentially of, or consists of the nucleic acid sequence CTCCATCACTAGGGGTTCCT (SEQ ID NO: 16).

In some embodiments, the D-sequence of an ITR (e.g., the first ITR or the second ITR) of a nucleic acid vector disclosed herein is entirely or partially removed. In some embodiments, the D-sequence of both ITRs of a nucleic acid vector disclosed herein is entirely or partially removed. In some embodiments, the D-sequence of an ITR (e.g., the first ITR or the second ITR) is entirely or partially replaced with a non-AAV sequence (i.e., a nucleotide sequence that is not from an AAV nucleic acid). In some embodiments, the D-sequence of an ITR (e.g., the first ITR or the second ITR) is entirely or partially replaced with an S-sequence. In some embodiments, the S-sequence comprises, consists essentially of, or consists of the nucleic acid sequence TATTAGATCTGATGGCCGCT (SEQ ID NO: 17). In some embodiments, the S-sequence has at least 70% identity (e.g., at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity) with the sequence TATTAGATCTGATGGCCGCT (SEQ ID NO: 17). In some embodiments, the S-sequence has less than 95% identity (e.g., less than 90% identity, less than 85% identity, less than 80% identity, less than 75% identity, or less than 70% identity) with the sequence TATTAGATCTGATGGCCGCT (SEQ ID NO: 17). In some embodiments, the S-sequence has about 70% to about 95% identity (e.g., about 95% identity, about 90% identity, about 85% identity, about 80% identity, about 75% identity, or about 70% identity) with the sequence TATTAGATCTGATGGCCGCT (SEQ ID NO: 17). In some embodiments, the S-sequence has fewer than 6 mismatches (e.g., fewer than 5, fewer than 4, fewer than 3, fewer than 2, 1, or no mismatches) relative to the sequence TATTAGATCTGATGGCCGCT (SEQ ID NO: 17). In some embodiments, the S-sequence has 1, 2, 3, 4, 5, or 6 mismatches relative to the sequence TATTAGATCTGATGGCCGCT (SEQ ID NO: 17). In some embodiments, the S-sequence has a length of or about 15, 16, 17, 18, 19, 20, 21, 22, 23, 24, or 25 nucleotides.

In some embodiments, the D-sequence of an ITR (e.g., the first ITR or the second ITR) is entirely or partially substituted with a glucocorticoid receptor-binding element (GRE). In some embodiments, a GRE is inserted into a nucleic acid vector (i.e., instead of substituting a portion of an ITR). For example, a GRE may be inserted inside the D-sequence of an ITR, upstream of the D-sequence of an ITR, or downstream of the D-sequence of an ITR.

Glucocorticoid receptor-binding elements are also known as glucocorticoid responsive elements or glucocorticoid response elements. GREs are nucleotide sequences that glucocorticoid receptor binds, which in their native loci are generally about 100 to 2,000 base pairs upstream from the transcription initiation site of a gene. The present disclosure is based in part on the discovery that a portion of the AAV2 D-sequence shares partial homology to the consensus half-site of the GRE, and that the glucocorticoid receptor signaling pathway is activated following AAV2 infection or transduction. In some embodiments, substitution of a portion or all of a D-sequence of an AAV ITR with a GRE increases expression of a transgene encoded by a nucleic acid vector encapsidated within an AAV particle.

In some embodiments, the GRE comprises, consists essentially of, or consists of at least 8 contiguous nucleotides (e.g., 8, 9, 10, 11, 12, 13, 14, or 15 contiguous nucleotides) of the nucleic acid sequence AGAACANNNTGTTCT (SEQ ID NO: 18), or its reverse or reverse complement, wherein each N is independently a T, C, G, or A. In some embodiments, the GRE has at least 70% identity (e.g., at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity) with at least 8 contiguous nucleotides (e.g., 8, 9, 10, 11, 12, 13, 14, or 15 contiguous nucleotides) of the sequence AGAACANNNTGTTCT (SEQ ID NO: 18), or its reverse or reverse complement, wherein each N is independently a T, C, G, or A. In some embodiments, the GRE has less than 95% identity (e.g., less than 90% identity, less than 85% identity, less than 80% identity, less than 75% identity, or less than 70% identity) with at least 8 contiguous nucleotides (e.g., 8, 9, 10, 11, 12, 13, 14, or 15 contiguous nucleotides) of the sequence AGAACANNNTGTTCT (SEQ ID NO: 18), or its reverse or reverse complement, wherein each N is independently a T, C, G, or A. In some embodiments, the GRE has about 70% to about 95% identity (e.g., about 95% identity, about 90% identity, about 85% identity, about 80% identity, about 75% identity, or about 70% identity) with at least 8 contiguous nucleotides (e.g., 8, 9, 10, 11, 12, 13, 14, or 15 contiguous nucleotides) of the sequence AGAACANNNTGTTCT (SEQ ID NO: 18), or its reverse or reverse complement, wherein each N is independently a T, C, G, or A. In some embodiments, the GRE has fewer than 6 mismatches (e.g., fewer than 5, fewer than 4, fewer than 3, fewer than 2, 1, or no mismatches) relative to the sequence AGAACANNNTGTTCT (SEQ ID NO: 18), or its reverse or reverse complement, wherein each N is independently a T, C, G, or A. In some embodiments, the GRE has 1, 2, 3, 4, 5, or 6 mismatches relative to the sequence AGAACANNNTGTTCT (SEQ ID NO: 18), or its reverse or reverse complement, wherein each N is independently a T, C, G, or A. In some embodiments, the GRE has a length of or about 8, 9, 10, 11, 12, 13, 14, 15, 16, 17, 18, 19, 20, 21, 22, 23, 24, or 25 nucleotides. In some embodiments, the GRE has a length of 15 nucleotides. In some embodiments, the GRE comprises, consists essentially of, or consists of the nucleic acid sequence AGAACANNNTGTTCT (SEQ ID NO: 18), or its reverse or reverse complement, wherein each N is independently a T, C, G, or A.

In some embodiments, the GRE comprises, consists essentially of, or consists of at least 8 contiguous nucleotides (e.g., 8, 9, 10, 11, 12, 13, 14, or 15 contiguous nucleotides) of the nucleic acid sequence GGTACANNNTGTYCT (SEQ ID NO: 19), or its reverse or reverse complement, wherein each N is independently a T, C, G, or A, and wherein Y is a T or C. In some embodiments, the GRE has at least 70% identity (e.g., at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity) with at least 8 contiguous nucleotides (e.g., 8, 9, 10, 11, 12, 13, 14, or 15 contiguous nucleotides) of the sequence GGTACANNNTGTYCT (SEQ ID NO: 19), or its reverse or reverse complement, wherein each N is independently a T, C, G, or A, and wherein Y is a T or C. In some embodiments, the GRE has less than 95% identity (e.g., less than 90% identity, less than 85% identity, less than 80% identity, less than 75% identity, or less than 70% identity) with at least 8 contiguous nucleotides (e.g., 8, 9, 10, 11, 12, 13, 14, or 15 contiguous nucleotides) of the sequence GGTACANNNTGTYCT (SEQ ID NO: 19), or its reverse or reverse complement, wherein each N is independently a T, C, G, or A, and wherein Y is a T or C. In some embodiments, the GRE has about 70% to about 95% identity (e.g., about 95% identity, about 90% identity, about 85% identity, about 80% identity, about 75% identity, or about 70% identity) with at least 8 contiguous nucleotides (e.g., 8, 9, 10, 11, 12, 13, 14, or 15 contiguous nucleotides) of the sequence GGTACANNNTGTYCT (SEQ ID NO: 19), or its reverse or reverse complement, wherein each N is independently a T, C, G, or A, and wherein Y is a T or C. In some embodiments, the GRE has fewer than 6 mismatches (e.g., fewer than 5, fewer than 4, fewer than 3, fewer than 2, 1, or no mismatches) relative to the sequence GGTACANNNTGTYCT (SEQ ID NO: 19), or its reverse or reverse complement, wherein each N is independently a T, C, G, or A, and wherein Y is a T or C. In some embodiments, the GRE has 1, 2, 3, 4, 5, or 6 mismatches relative to the sequence GGTACANNNTGTYCT (SEQ ID NO: 19), or its reverse or reverse complement, wherein each N is independently a T, C, G, or A, and wherein Y is a T or C. In some embodiments, the GRE has a length of or about 8, 9, 10, 11, 12, 13, 14, 15, 16, 17, 18, 19, 20, 21, 22, 23, 24, or 25 nucleotides. In some embodiments, the GRE has a length of 15 nucleotides. In some embodiments, the GRE comprises, consists essentially of, or consists of the nucleic acid sequence GGTACANNNTGTYCT (SEQ ID NO: 19), or its reverse or reverse complement, wherein each N is independently a T, C, G, or A, and wherein Y is a T or C.

In some embodiments, the GRE comprises, consists essentially of, or consists of at least 8 contiguous nucleotides (e.g., 8, 9, 10, 11, 12, 13, 14, or 15 contiguous nucleotides) of the nucleic acid sequence AGAACAGGATGTTCT (SEQ ID NO: 20), or its reverse or reverse complement. In some embodiments, the GRE has at least 70% identity (e.g., at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity) with at least 8 contiguous nucleotides (e.g., 8, 9, 10, 11, 12, 13, 14, or 15 contiguous nucleotides) of the sequence AGAACAGGATGTTCT (SEQ ID NO: 20), or its reverse or reverse complement. In some embodiments, the GRE has less than 95% identity (e.g., less than 90% identity, less than 85% identity, less than 80% identity, less than 75% identity, or less than 70% identity) with at least 8 contiguous nucleotides (e.g., 8, 9, 10, 11, 12, 13, 14, or 15 contiguous nucleotides) of the sequence AGAACAGGATGTTCT (SEQ ID NO: 20), or its reverse or reverse complement. In some embodiments, the GRE has about 70% to about 95% identity (e.g., about 95% identity, about 90% identity, about 85% identity, about 80% identity, about 75% identity, or about 70% identity) with at least 8 contiguous nucleotides (e.g., 8, 9, 10, 11, 12, 13, 14, or 15 contiguous nucleotides) of the sequence AGAACAGGATGTTCT (SEQ ID NO: 20), or its reverse or reverse complement. In some embodiments, the GRE has fewer than 6 mismatches (e.g., fewer than 5, fewer than 4, fewer than 3, fewer than 2, 1, or no mismatches) relative to the sequence AGAACAGGATGTTCT (SEQ ID NO: 20), or its reverse or reverse complement. In some embodiments, the GRE has 1, 2, 3, 4, 5, or 6 mismatches relative to the sequence AGAACAGGATGTTCT (SEQ ID NO: 20), or its reverse or reverse complement. In some embodiments, the GRE has a length of or about 8, 9, 10, 11, 12, 13, 14, 15, 16, 17, 18, 19, 20, 21, 22, 23, 24, or 25 nucleotides. In some embodiments, the GRE has a length of 15 nucleotides. In some embodiments, the GRE comprises, consists essentially of, or consists of the nucleic acid sequence AGAACAGGATGTTCT (SEQ ID NO: 20), or its reverse or reverse complement.

Another example of a GRE sequence useful in accordance with the present disclosure is 5′-GGCACAGTGTGGTCT-3′ (SEQ ID NO: 21). Other GRE sequences can be used, including for example GRE sequences that are known in the art.

In some embodiments, substitution of a D-sequence comprises substitution of at least 5 nucleotides (e.g., 5, 6, 7, 8, 9, 10, 11, 12, 13, 14, 15, 16, 17, 18, 19, or 20 nucleotides) of the D-sequence with a different nucleotide sequence (e.g., an S-sequence or portion thereof, or a GRE or portion thereof). In some embodiments, substitution of a D-sequence comprises substitution of 10 nucleotides of the D-sequence. In some embodiments, substitution of a D-sequence comprises substitution of the 3′-most 10 nucleotides of the D-sequence. In some embodiments, substitution of a D-sequence comprises substitution of the 5′-most 10 nucleotides of the D-sequence. In some embodiments, substitution of a D-sequence comprises substitution of an internal portion (i.e., not comprising a terminal nucleotide) of the D-sequence, such as 10 nucleotides of the internal portion of the D-sequence.

In some embodiments, deletion of a D-sequence comprises deletion of at least 5 nucleotides (e.g., 5, 6, 7, 8, 9, 10, 11, 12, 13, 14, 15, 16, 17, 18, 19, or 20 nucleotides) of the D-sequence. In some embodiments, deletion of a D-sequence comprises deletion of 10 nucleotides of the D-sequence. In some embodiments, deletion of a D-sequence comprises deletion of the 3′-most 10 nucleotides of the D-sequence. In some embodiments, deletion of a D-sequence comprises deletion of the 5′-most 10 nucleotides of the D-sequence. In some embodiments, deletion of a D-sequence comprises deletion of an internal portion (i.e., not comprising a terminal nucleotide) of the D-sequence, such as 10 nucleotides of the internal portion of the D-sequence.

A nucleic acid vector as disclosed herein in some embodiments comprises one or more regulatory elements. A regulatory element refers to a nucleotide sequence or structural component of a nucleic acid vector which is involved in the regulation of expression of components of the nucleic acid vector (e.g., a gene of interest comprised therein). Regulatory elements include, but are not limited to, promoters, enhancers, silencers, insulators, response elements, initiation sites, termination signals, and ribosome binding sites.

Promoters include constitutive promoters, inducible promoters, tissue-specific promoters, cell type-specific promoters, and synthetic promoters. For example, a nucleic acid vector disclosed herein may include viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter.

Inducible promoters or other inducible regulatory elements may also be used to achieve desired expression levels of a gene of interest (e.g., a protein or polypeptide of interest). Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters or other tissue-specific regulatory elements are also contemplated herein. Non-limiting examples of such promoters that may be used include muscle-specific promoters.

Synthetic promoters are also contemplated herein. A synthetic promoter may comprise, for example, regions of known promoters, regulatory elements, transcription factor binding sites, enhancer elements, repressor elements, and the like.

In some embodiments, a nucleic acid provided herein comprises a nucleotide sequence encoding a product (e.g., a protein or polypeptide product). In some embodiments, a nucleotide sequence comprises a nucleotide sequence of a gene of interest. In some embodiments, a gene of interest encodes a therapeutic or diagnostic protein or polypeptide. In some embodiments, a therapeutic or diagnostic protein or polypeptide is an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic agent, an enzyme, a bone morphogenetic protein, a nuclease, a protein used for gene editing, an Fc-fusion protein, an anticoagulant, or a protein or polypeptide that can be detected using a laboratory test. In some embodiments, a nucleic acid provided herein comprises a nucleotide sequence encoding a guide RNA or other nucleic acid used for gene editing, optionally in addition to a protein used for gene editing.

In some embodiments, a product encoded by a nucleic acid disclosed herein is a detectable molecule. A detectable molecule is a molecule that can be visualized (e.g., using a naked eye, under a microscope, or using a light detection device such as a camera). In some embodiments, the detectable molecule is a fluorescent molecule, a bioluminescent molecule, or a molecule that provides color (e.g., β-galactosidase, β-lactamase, β-glucuronidase, or spheroidenone). In some embodiments, the detectable molecule is a fluorescent, bioluminescent or enzymatic protein or functional peptide or polypeptide thereof.

In some embodiments, fluorescent protein is a blue fluorescent protein, a cyan fluorescent protein, a green fluorescent protein, a yellow fluorescent protein, an orange fluorescent protein, a red fluorescent protein, or a functional peptide or polypeptide thereof. A blue fluorescent protein may be azurite, EBFP, EBFP2, mTagBFP, or Y66H. A cyan fluorescent protein may be ECFP, AmCyan1, Cerulean, CyPet, mECFP, Midori-ishi Cyan, mTFP1, or TagCFP. A Green fluorescent protein may be AcGFP, Azami Green, EGFP, Emarald, GFP or a mutated form of GFP (e.g., GFP-S65T, mWasabi, Stemmer, Superfolder GFP, TagGFP, TurboGFP, or ZsGreen). A yellow fluorescent protein may be EYFP, mBanana, mCitrine, PhiYFp, TagYFP, Topaz, Venus, YPet, or ZsYellow1. An orange fluorescent protein may be DsRed, RFP, DsRed2, DsRed-Express, Ds-Red-monomer, Tomato, tdTomato, Kusabira Orange, mKO2, mOrange, mOrange2, mTangerine, TagRFP, or TagRFP-T. A red fluorescent protein may be AQ142, AsRed2, dKeima-Tandem, HcRed1, tHcRed, Jred, mApple, mCherry, mPlum, mRasberry, mRFP1, mRuby or mStrawberry.

In some embodiments, a detectable molecule is a bioluminescent protein or a functional peptide or polypeptide thereof. Non-limiting examples of bioluminescent proteins are firefly luciferase, click-beetle luciferase, Renilla luciferase, and luciferase from Oplophorus gracilirostris.

In some embodiments, a detectable molecule may be any polypeptide or protein that can be detected using methods known in the art. Non-limiting methods of detection are fluorescence imaging, luminescent imaging, bright filed imaging, and include imaging facilitated by immunofluorescence or immunohistochemical staining.

Additional features of AAV particles, nucleic acid vectors, and capsid proteins are described in U.S. Patent Publication No. 2017/0356009, the contents of which are incorporated herein by reference in their entirety.

### AAV Particles

According to some aspects, AAV particles are provided herein. An AAV particle is a supramolecular assembly of 60 individual capsid protein subunits forming a non-enveloped T-1 icosahedral lattice capable of protecting a 4.7-kb single-stranded DNA genome. A mature AAV particle is approximately 20 nm in diameter, and its capsid is formed from three structural capsid proteins VP1, VP2, and VP3, with molecular masses of 87, 73, and 62 kDa, respectively, in a ratio of approximately 1:1:18. The 60 capsid proteins are arranged in an anti-parallel β-strand barreloid arrangement, resulting in a defined tropism and a high resistance to degradation.

In some embodiments, an AAV particle comprises an empty capsid (e.g., a capsid without a cargo). In some embodiments, an AAV particle comprises a capsid encapsidating a nucleic acid (e.g., a nucleic acid vector that comprises a gene of interest, such as a nucleic acid vector disclosed herein). In some embodiments, a nucleic acid encapsidated within an AAV capsid to generate an AAV particle comprises a nucleic acid vector disclosed herein. In some embodiments, an AAV particle disclosed herein comprises a capsid protein comprising one or more mutations, e.g., one or more amino acid substitutions.

It is contemplated herein that any capsid protein mutations disclosed herein (e.g., amino acid substitutions) can be combined with any nucleic acid vector modifications disclosed herein (e.g., sequence deletions or substitutions). For example, an AAV particle described herein may have an AAVrh74 capsid protein (e.g., a wild-type AAVrh74 capsid protein or one comprising one or more amino acid substitutions) and an AAV nucleic acid vector (e.g., an AAV2 nucleic acid vector) comprising a modification (e.g., a deletion or substitution of a D-sequence, and/or an insertion of a non-AAV sequence, such as a GRE).

In some embodiments, an AAV particle disclosed herein comprises a capsid protein comprising amino acid substitutions at one or more positions corresponding to Y447, T494, K547, N665, and Y733 of the wild-type AAVrh74 capsid protein of SEQ ID NO: 1. In some embodiments, an AAV particle disclosed herein comprises a capsid protein comprising one or more amino acid substitutions corresponding to Y447F, T494V, K547R, N665R, and Y733F of the wild-type AAVrh74 capsid protein of SEQ ID NO: 1.

In some embodiments, an AAV particle disclosed herein comprises a capsid protein comprising amino acid substitutions at one or more positions corresponding to Y447, T494, K547, N665, and Y733 of the wild-type AAVrh74 capsid protein of SEQ ID NO: 1 and further comprises a nucleic acid vector comprising modification (e.g., a deletion or a substitution) of a D-sequence of an ITR (e.g., a modification of a D-sequence of a right ITR, a left ITR, or both a right ITR and a left ITR).

In some embodiments, the AAV particle comprises a capsid protein comprising amino acid substitutions at one or more positions corresponding to Y447, T494, K547, N665, and Y733 of the wild-type AAVrh74 capsid protein of SEQ ID NO: 1 and a nucleic acid vector comprising a substitution of a D-sequence of an ITR with an S-sequence. In some embodiments, the amino acid substitutions correspond to Y447F, T494V, K547R, N665R, and Y733F of the wild-type AAVrh74 capsid protein of SEQ ID NO: 1. In some embodiments, the S-sequence comprises, consists essentially of, or consists of the nucleotide sequence TATTAGATCTGATGGCCGCT (SEQ ID NO: 17). In some embodiments, a portion or the entirety of a D-sequence of an ITR (e.g., the D-sequence of the left ITR) is substituted with the S-sequence.

In some embodiments, the AAV particle comprises a capsid protein comprising amino acid substitutions corresponding to Y447F and Y733F of the wild-type AAVrh74 capsid protein of SEQ ID NO: 1 and a nucleic acid vector comprising a substitution of a D-sequence of an ITR with an S-sequence. In some embodiments, the S-sequence comprises, consists essentially of, or consists of the nucleotide sequence TATTAGATCTGATGGCCGCT (SEQ ID NO: 17). In some embodiments, a portion or the entirety of a D-sequence of an ITR (e.g., the D-sequence of the left ITR) is substituted with the S-sequence.

In some embodiments, the AAV particle comprises a capsid protein comprising amino acid substitutions corresponding to Y447F, T494V, and Y733F of the wild-type AAVrh74 capsid protein of SEQ ID NO: 1 and a nucleic acid vector comprising a substitution of a D-sequence of an ITR with an S-sequence. In some embodiments, the S-sequence comprises, consists essentially of, or consists of the nucleotide sequence TATTAGATCTGATGGCCGCT (SEQ ID NO: 17). In some embodiments, a portion or the entirety of a D-sequence of an ITR (e.g., the D-sequence of the left ITR) is substituted with the S-sequence.

In some embodiments, the AAV particle comprises a capsid protein comprising amino acid substitutions at one or more positions corresponding to Y447, T494, K547, N665, and Y733 of the wild-type AAVrh74 capsid protein of SEQ ID NO: 1 and a nucleic acid vector comprising a deletion of all or a portion of a D-sequence of an ITR of the nucleic acid vector. In some embodiments, the amino acid substitutions correspond to Y447F and Y733F of the wild-type AAVrh74 capsid protein of SEQ ID NO: 1. In some embodiments, the amino acid substitutions correspond to Y447F, T494V, and Y733F of the wild-type AAVrh74 capsid protein of SEQ ID NO: 1. In some embodiments, the amino acid substitutions correspond to Y447F, T494V, K547R, N665R, and Y733F of the wild-type AAVrh74 capsid protein of SEQ ID NO: 1.

In some embodiments, the AAV particle comprises a capsid protein comprising amino acid substitutions at one or more positions corresponding to Y447, T494, K547, N665, and Y733 of the wild-type AAVrh74 capsid protein of SEQ ID NO: 1 and a nucleic acid vector comprising a substitution of a D-sequence of an ITR with a GRE. In some embodiments, the amino acid substitutions correspond to Y447F, T494V, K547R, N665R, and Y733F of the wild-type AAVrh74 capsid protein of SEQ ID NO: 1. In some embodiments, the GRE comprises, consists essentially of, or consists of the nucleic acid sequence GGTACANNNTGTYCT (SEQ ID NO: 19), or its reverse or reverse complement, wherein each N is independently a T, C, G, or A, and wherein Y is a T or C. In some embodiments, the GRE comprises, consists essentially of, or consists of the nucleic acid sequence AGAACANNNTGTTCT (SEQ ID NO: 18), or its reverse or reverse complement, wherein each N is independently a T, C, G, or A. In some embodiments, the GRE comprises, consists essentially of, or consists of the nucleic acid sequence AGAACAGGATGTTCT (SEQ ID NO: 20), or its reverse or reverse complement. In some embodiments, a portion or the entirety of a D-sequence of an ITR (e.g., the D-sequence of the left ITR) is substituted with the GRE.

In some embodiments, the AAV particle comprises a capsid protein comprising amino acid substitutions corresponding to Y447F and Y733F of the wild-type AAVrh74 capsid protein of SEQ ID NO: 1 and a nucleic acid vector comprising a substitution of a D-sequence of an ITR with a GRE. In some embodiments, the GRE comprises, consists essentially of, or consists of the nucleic acid sequence GGTACANNNTGTYCT (SEQ ID NO: 19), or its reverse or reverse complement, wherein each N is independently a T, C, G, or A, and wherein Y is a T or C. In some embodiments, the GRE comprises, consists essentially of, or consists of the nucleic acid sequence AGAACANNNTGTTCT (SEQ ID NO: 18), or its reverse or reverse complement, wherein each N is independently a T, C, G, or A. In some embodiments, the GRE comprises, consists essentially of, or consists of the nucleic acid sequence AGAACAGGATGTTCT (SEQ ID NO: 20), or its reverse or reverse complement. In some embodiments, a portion or the entirety of a D-sequence of an ITR (e.g., the D-sequence of the left ITR) is substituted with the GRE.

In some embodiments, the AAV particle comprises a capsid protein comprising amino acid substitutions corresponding to Y447F, T494V, and Y733F of the wild-type AAVrh74 capsid protein of SEQ ID NO: 1 and a nucleic acid vector comprising a substitution of a D-sequence of an ITR with a GRE. In some embodiments, the GRE comprises, consists essentially of, or consists of the nucleic acid sequence GGTACANNNTGTYCT (SEQ ID NO: 19), or its reverse or reverse complement, wherein each N is independently a T, C, G, or A, and wherein Y is a T or C. In some embodiments, the GRE comprises, consists essentially of, or consists of the nucleic acid sequence AGAACANNNTGTTCT (SEQ ID NO: 18), or its reverse or reverse complement, wherein each N is independently a T, C, G, or A. In some embodiments, the GRE comprises, consists essentially of, or consists of the nucleic acid sequence AGAACAGGATGTTCT (SEQ ID NO: 20), or its reverse or reverse complement. In some embodiments, a portion or the entirety of a D-sequence of an ITR (e.g., the D-sequence of the left ITR) is substituted with the GRE.

In some embodiments, an AAV particle disclosed herein is replicative. A replicative AAV particle is capable of replicating within a host cell (e.g., a host cell within a subject or a host cell in culture). In some embodiments, an AAV particle disclosed herein is non-replicating. A non-replicating AAV particle is not capable of replicating within a host cell (e.g., a host cell within a subject or a host cell in culture), but can infect the host and incorporate a genetic components into the host's genome for expression. In some embodiments, an AAV particle disclosed herein is capable of infecting a host cell. In some embodiments, an AAV particle disclosed herein is capable of facilitating stable integration of genetic components into the genome of a host cell. In some embodiments, an AAV particle disclosed herein is not capable of facilitating integration of genetic components into the genome of a host cell.

In some embodiments, an AAV particle disclosed herein comprises a nucleic acid vector. In some embodiments, a nucleic acid vector comprises two inverted terminal repeats (ITRs) adjacent to the ends of a sequence encoding a gene of interest. In some embodiments, the nucleic acid vector is comprised within the AAV's ssDNA genome. In some embodiments, an AAV particle disclosed herein comprises one single-stranded DNA. In some embodiments, an AAV particle disclosed herein comprises two complementary DNA strands, forming a self-complementary AAV (scAAV).

In some embodiments, a nucleic acid vector that may be comprised in an AAV particle (e.g., a WT particle or particle comprising a capsid comprising any one or more mutations as disclosed herein) comprises an ITR comprising a modification (e.g., a deletion or substitution) of part or all of the ITR's D-sequence. In some embodiments, part or all of the ITR's D-sequence is substituted with an S-sequence or a portion thereof. In some embodiments, part or all of the ITR's D-sequence is substituted with a GRE or a portion thereof. In some embodiments, part or all of the ITR's D-sequence is deleted. Further description of such modifications (e.g., deletions and substitutions) is provided elsewhere herein.

An AAV particle disclosed herein may be of any AAV serotype (e.g., AAV serotype 1, 2, 3, 4, 5, 6, 7, 8, 9, 10, 11, 12, or 13), including any derivative (including non-naturally occurring variants of a serotype) or pseudotype. Non-limiting examples of derivatives and pseudotypes include AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV2.5T, AAV-HAE1/2, AAV clone 32/83, AAVShH10, AAV2.15, AAV2.4, AAVM41, and AAVr3.45. Such AAV serotypes and derivatives/pseudotypes, and methods of producing such derivatives/pseudotypes are known in the art (see, e.g., Mol. Ther. 2012 Apr; 20(4):699-708. doi: 10.1038/mt.2011.287. Epub 2012 Jan 24. The AAV vector toolkit: poised at the clinical crossroads. Asokan A, Schaffer DV, Samulski RJ.). In some embodiments, the AAV particle is a pseudotyped AAV particle, which comprises a nucleic acid vector comprising ITRs from one serotype (e.g., AAV2 or AAV3) and a capsid comprised of capsid proteins derived from another serotype (i.e., a serotype other than AAV2 or AAV3, respectively). Methods for producing and using pseudotyped rAAV vectors are known in the art (see, e.g., Duan et al., J. Virol., 75:7662-7671 (2001); Halbert et al., J. Virol., 74:1524-1532 (2000); Zolotukhin et al., Methods, 28:158-167 (2002); and Auricchio et al., Hum. Molec. Genet., 10:3075-3081 (2001)).

In some embodiments, an AAV particle disclosed herein is a recombinant AAV (rAAV) particle, e.g., comprising a recombinant nucleic acid or transgene.

Any combination of modifications described herein (e.g., capsid protein modifications, a deletion or substitution of a D-sequence, and/or an insertion of a non-AAV sequence into an AAV genome) may result in an additive or synergistic effect, in which the beneficial properties of the resulting combination are equal to or greater than, respectively, the sum of the effects of the individual modifications. For example, an AAV particle comprising a modified capsid protein and a modified genome may have improvements in transduction efficiency, transgene expression, and/or packaging efficiency relative to a corresponding wild-type AAV particle that are equal to the sum of the improvements conferred by the individual capsid protein modification and the genome modification, or that are greater than the sum of the improvements conferred by the individual modifications.

### Transduction Efficiency

According to some aspects, transduction efficiency of an AAV particle disclosed herein is modified relative to a corresponding wild-type AAV particle. Transduction efficiency of an AAV particle can be determined, for example, by comparing expression of a gene of interest in a cell following contacting the cell with the AAV particle, or by measuring the number of viral genome copies per cell following contacting a population of cells with the AAV particle. In some embodiments, transduction efficiency of an AAV particle as disclosed herein (e.g., an AAV particle comprising a modified capsid protein (e.g., comprising one or more amino acid substitutions), a modified nucleic acid vector (e.g., modified by deletion and/or substitution of a D-sequence), or both a modified capsid protein (e.g., comprising one or more amino acid substitutions) and a modified nucleic acid vector (e.g., modified by deletion and/or substitution of a D-sequence)) is higher than the transduction efficiency of a corresponding wild-type AAV particle. In some embodiments, the transduction efficiency of an AAV particle as disclosed herein is at least 5% higher (e.g., at least 10% higher, at least 15% higher, at least 20% higher, at least 25% higher, at least 30% higher, at least 35% higher, at least 40% higher, at least 50% higher, at least 60% higher, at least 70% higher, at least 80% higher, at least 90% higher, at least 100% higher, at least 150% higher, at least 200% higher, at least 250% higher, or more) than the transduction efficiency of a corresponding wild-type AAV particle. In some embodiments, the transduction efficiency of an AAV particle as disclosed herein is at least 1.5-fold higher (e.g., at least 2-fold higher, at least 2.5-fold higher, at least 3-fold higher, at least 3.5-fold higher, at least 4-fold higher, at least 4.5-fold higher, at least 5-fold higher, at least 5.5-fold higher, at least 6-fold higher, at least 6.5-fold higher, at least 7-fold higher, at least 7.5-fold higher, at least 8-fold higher, at least 8.5-fold higher, at least 9-fold higher, at least 9.5-fold higher, at least 10-fold higher, at least 10.5-fold higher, at least 11-fold higher, at least 11.5-fold higher, at least 12-fold higher, at least 12.5-fold higher, at least 13-fold higher, at least 13.5-fold higher, at least 14-fold higher, at least 14.5-fold higher, at least 15-fold higher, at least 15.5-fold higher, at least 16-fold higher, at least 16.5-fold higher, at least 17-fold higher, at least 17.5-fold higher, at least 18-fold higher, at least 18.5-fold higher, at least 19-fold higher, at least 19.5-fold higher, at least 20-fold higher, or more) than the transduction efficiency of a corresponding wild-type AAV particle. In some embodiments, transduction efficiency of an AAV particle as disclosed herein is not modified relative to a corresponding wild-type AAV particle.

### Transgene Expression

According to some aspects, expression of a transgene encoded by a nucleic acid vector comprising a modification (e.g., a deletion or substitution of a sequence, such as a D-sequence) disclosed herein is altered relative to expression of the transgene encoded by a nucleic acid vector that does not comprise the modification. Such alteration of transgene expression is, in some embodiments, on a per nucleic acid vector copy number basis (e.g., transgene expression in a cell, when normalized to the total amount of nucleic acid vector in the cell, is altered). For example, in some embodiments, a modified AAV particle as disclosed herein results in greater transgene expression relative to a corresponding AAV particle not comprising the same modification but that delivers a comparable number of viral genomes to a cell. Relative transgene expression levels can be determined, for example, by measuring expression of the transgene in a cell by methods known in the art following contacting the cell with an AAV particle comprising the modified nucleic acid vector encoding the transgene and comparing an equivalent measurement in another cell contacted with an AAV particle comprising a nucleic acid vector that does not comprise the modification.

In some embodiments, transgene expression from a modified nucleic acid vector as disclosed herein (e.g., modified by deletion and/or substitution of a D-sequence) is higher than the transgene expression from a corresponding nucleic acid vector that does not comprise the modification. In some embodiments, the transgene expression from a modified nucleic acid vector as disclosed herein is at least 5% higher (e.g., at least 10% higher, at least 15% higher, at least 20% higher, at least 25% higher, at least 30% higher, at least 35% higher, at least 40% higher, at least 50% higher, at least 60% higher, at least 70% higher, at least 80% higher, at least 90% higher, at least 100% higher, at least 150% higher, at least 200% higher, at least 250% higher, or more) than the transgene expression from a corresponding nucleic acid vector that does not comprise the modification.

In some embodiments, the transgene expression from a modified nucleic acid vector as disclosed herein is at least 1.5-fold higher (e.g., at least 2-fold higher, at least 2.5-fold higher, at least 3-fold higher, at least 3.5-fold higher, at least 4-fold higher, at least 4.5-fold higher, at least 5-fold higher, at least 5.5-fold higher, at least 6-fold higher, at least 6.5-fold higher, at least 7-fold higher, at least 7.5-fold higher, at least 8-fold higher, at least 8.5-fold higher, at least 9-fold higher, at least 9.5-fold higher, at least 10-fold higher, at least 10.5-fold higher, at least 11-fold higher, at least 11.5-fold higher, at least 12-fold higher, at least 12.5-fold higher, at least 13-fold higher, at least 13.5-fold higher, at least 14-fold higher, at least 14.5-fold higher, at least 15-fold higher, at least 15.5-fold higher, at least 16-fold higher, at least 16.5-fold higher, at least 17-fold higher, at least 17.5-fold higher, at least 18-fold higher, at least 18.5-fold higher, at least 19-fold higher, at least 19.5-fold higher, at least 20-fold higher, or more) than the transgene expression from a corresponding nucleic acid vector that does not comprise the modification.

In some embodiments, transgene expression from a modified nucleic acid vector as disclosed herein is not changed relative to transgene expression from a corresponding nucleic acid vector that does not comprise the modification.

### Packaging Efficiency

According to some aspects, packaging efficiency of an AAV particle disclosed herein is modified relative to a corresponding wild-type AAV particle. Packaging efficiency of an AAV particle refers to the capability of a particular AAV capsid to encapsidate a particular viral genome. Packaging efficiency can be measured by one of ordinary skill in the art, such as by quantifying the ratio of capsids to viral genomes (sec, e.g., Grimm, et al. Gene Ther. 6:1322-1330 (1999)).

In some embodiments, the packaging efficiency of an AAV particle as disclosed herein (e.g., an AAV particle comprising a modified capsid protein, a modified nucleic acid vector, or both a modified capsid protein and a modified nucleic acid vector) is higher than the packaging efficiency of a corresponding wild-type AAV particle. In some embodiments, the packaging efficiency of an AAV particle as disclosed herein is at least 5% higher (e.g., at least 10% higher, at least 15% higher, at least 20% higher, at least 25% higher, at least 30% higher, at least 35% higher, at least 40% higher, at least 50% higher, at least 60% higher, at least 70% higher, at least 80% higher, at least 90% higher, at least 100% higher, at least 150% higher, at least 200% higher, at least 250% higher, or more) than the packaging efficiency of a corresponding wild-type AAV particle. In some embodiments, the packaging efficiency of an AAV particle as disclosed herein is at least 1.5-fold higher (e.g., at least 2-fold higher, at least 2.5-fold higher, at least 3-fold higher, at least 3.5-fold higher, at least 4-fold higher, at least 4.5-fold higher, at least 5-fold higher, at least 5.5-fold higher, at least 6-fold higher, at least 6.5-fold higher, at least 7-fold higher, at least 7.5-fold higher, at least 8-fold higher, at least 8.5-fold higher, at least 9-fold higher, at least 9.5-fold higher, at least 10-fold higher, at least 10.5-fold higher, at least 11-fold higher, at least 11.5-fold higher, at least 12-fold higher, at least 12.5-fold higher, at least 13-fold higher, at least 13.5-fold higher, at least 14-fold higher, at least 14.5-fold higher, at least 15-fold higher, at least 15.5-fold higher, at least 16-fold higher, at least 16.5-fold higher, at least 17-fold higher, at least 17.5-fold higher, at least 18-fold higher, at least 18.5-fold higher, at least 19-fold higher, at least 19.5-fold higher, at least 20-fold higher, or more) than the packaging efficiency of a corresponding wild-type AAV particle.

In some embodiments, the packaging efficiency of an AAV particle as disclosed herein (e.g., an AAV particle comprising a modified capsid protein, a modified nucleic acid vector, or both a modified capsid protein and a modified nucleic acid vector) is lower than the packaging efficiency of a corresponding wild-type AAV particle. In some embodiments, the packaging efficiency of an AAV particle as disclosed herein is decreased by at least 5% (e.g., at least 10%, at least 15%, at least 20%, at least 25%, at least 30%, at least 35%, at least 40%, at least 50%, at least 60%, at least 70%, at least 75%, at least 80%, at least 85%, at least 90%, at least 91%, at least 92%, at least 93%, at least 94%, at least 95%, or more) relative to the packaging efficiency of a corresponding wild-type AAV particle.

In some embodiments, packaging efficiency of an AAV particle disclosed herein is not modified relative to a corresponding wild-type AAV particle.

In some embodiments, both the transduction efficiency and the packaging is efficiency of an AAV particle as disclosed herein is modified (i.e., increased or decreased) relative to a corresponding unmodified or wild-type AAV particle (e.g., of the same serotype). In some embodiments, the immunogenicity of an AAV particle as disclosed herein is modified relative to a corresponding unmodified or wild-type AAV particle (e.g., of the same serotype).

### Pharmaceutical Compositions

Any one of the AAV particles, capsid proteins, or nucleic acids disclosed herein may be comprised within a pharmaceutical composition comprising a pharmaceutically-acceptable carrier or may be comprised within a pharmaceutically-acceptable carrier. The term “carrier” refers to a diluent, adjuvant, excipient, or vehicle with which the AAV particle, capsid protein, or nucleic acid is comprised or administered to a subject. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases), and solutions or compositions thereof. Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of AAV particles to human subjects.

Typically, such compositions may contain at least about 0.1% of the therapeutic agent (e.g., AAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., AAV particle) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be designed.

### Methods of Contacting a Cell

According to some aspects, methods of contacting a cell with an AAV particle are provided herein. Methods of contacting a cell may comprise, for example, contacting a cell in a culture with a composition comprising an AAV particle. In some embodiments, contacting a cell comprises adding a composition comprising an AAV particle to the supernatant of a cell culture (e.g., a cell culture on a tissue culture plate or dish) or mixing a composition comprising an AAV particle with a cell culture (e.g., a suspension cell culture). In some embodiments, contacting a cell comprises mixing a composition comprising an AAV particle with another solution, such as a cell culture media, and incubating a cell with the mixture.

In some embodiments, contacting a cell with an AAV particle comprises administering a composition comprising an AAV particle to a subject or device in which the cell is located. In some embodiments, contacting a cell comprises injecting a composition comprising an AAV particle into a subject in which the cell is located. In some embodiments, contacting a cell comprises administering a composition comprising an AAV particle directly to a cell, or into or substantially adjacent to a tissue of a subject in which the cell is present.

In some embodiments, “administering” or “administration” means providing a material to a subject in a manner that is pharmacologically useful. In some embodiments, a rAAV particle is administered to a subject enterally. In some embodiments, an enteral administration of the essential metal element/s is oral. In some embodiments, a rAAV particle is administered to the subject parenterally. In some embodiments, a rAAV particle is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a rAAV particle is administered to the subject by injection into the hepatic artery or portal vein.

In some embodiments, a compositions of AAV particles is administered to a subject to treat a disease or condition. To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host organ, tissue, or cell. A therapeutically acceptable amount may be an amount that is capable of treating a disease, e.g., a muscular dystrophy. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, a cell disclosed herein is a cell isolated or derived from a subject. In some embodiments, a cell is a mammalian cell (e.g., a cell isolated or derived from a mammal). In some embodiments, a cell is a human cell. In some embodiments, a cell is isolated or derived from a particular tissue of a subject, such as muscle tissue. In some embodiments, a cell is a muscle cell. In some embodiments, a cell is a skeletal muscle cell or a smooth muscle cell. In some embodiments, a cell is in vitro. In some embodiments, a cell is ex vivo. In some embodiments, a cell in in vivo. In some embodiments, a cell is within a subject (e.g., within a tissue or organ of a subject). In some embodiments, a cell is a primary cell. In some embodiments, a cell is from a cell line (e.g., an immortalized cell line). In some embodiments a cell is a cancer cell or an immortalized cell.

In some embodiments, “administering” or “administration” means providing a material to a subject in a manner that is pharmacologically useful.

In certain circumstances it will be desirable to deliver an AAV particle disclosed herein in a suitably formulated pharmaceutical composition disclosed herein either subcutaneously, intraocularly, intravitreally, subretinally, parenterally, intravenously (IV), intracerebro-ventricularly, intramuscularly, intrathecally (IT), intracisternally, orally, intraperitoneally, by oral or nasal inhalation, or by direct injection to one or more cells, tissues, or organs by direct injection. In some embodiments, the administration is a route suitable for systemic delivery, such as by intravenous injection. In some embodiments, the administration is a route suitable for local delivery, such as by intramuscular injection. In some embodiments, “administering” or “administration” means providing a material to a subject in a manner that is pharmacologically useful.

In some embodiments, the concentration of AAV particles administered to a subject may be on the order ranging from 10⁶ to 10¹⁴ particles/ml or 10³ to 10¹⁵ particles/ml, or any values therebetween for either range, such as for example, about 10⁶, 10⁷, 10⁸, 10⁹, 10¹⁰, 10¹¹, 10¹², 10¹³, or 10¹⁴ particles/ml. In some embodiments, AAV particles of a higher concentration than 10¹³ particles/ml are administered. In some embodiments, the concentration of AAV particles administered to a subject may be on the order ranging from 10⁶ to 10¹⁴ vector genomes (vgs)/ml or 10³ to 10¹⁵ vgs/ml, or any values therebetween for either range (e.g., 10⁶, 10⁷, 10⁸, 10⁹, 10¹⁰, 10¹¹, 10¹², 10¹³, or 10¹⁴ vgs/ml). In some embodiments, AAV particles of higher concentration than 10¹³ vgs/ml are administered. The AAV particles can be administered as a single dose, or divided into two or more administrations as may be required to achieve therapy of the particular disease or disorder being treated. In some embodiments, 0.0001 ml to 10 ml are delivered to a subject. In some embodiments, the number of AAV particles administered to a subject may be on the order ranging from 10⁶-10¹⁴ vgs/kg body mass of the subject, or any values therebetween (e.g., 10⁶, 10⁷, 10⁸, 10⁹, 10¹⁰, 10¹¹, 10¹², 10¹³, or 10¹⁴ vgs/kg). In some embodiments, the dose of AAV particles administered to a subject may be on the order ranging from 10¹²-10¹⁴ vgs/kg. In some embodiments, the volume of AAVrh74 composition delivered to a subject (e.g., via one or more routes of administration as described herein) is 0.0001 ml to 10 ml.

In some embodiments, a composition disclosed herein (e.g., comprising an AAV particle) is administered to a subject once. In some embodiments, the composition is administered to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a disease, disorder, or condition in the subject.

### Subjects

Aspects of the disclosure relate to methods for use with a subject, such as human or non-human primate subjects; with a host cell in situ in a subject; or with a host cell derived from a subject (e.g., ex vivo or in vitro). Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease or disorder that may be treated with gene therapy. In some embodiments, the subject has or is suspected of having a muscle disease or disorder. A muscle disease or disorder is typically characterized by one or more mutation(s) in the genome that results in abnormal structure or function of one or more proteins associated with muscle development, health, maintenance and/or function. Exemplary muscle disease and disorders include amyotrophic lateral sclerosis, Charcot-Marie-Tooth disease, multiple sclerosis, muscular dystrophy (e.g., Duchenne muscular dystrophy, facioscapulohumeral muscular dystrophy, Becker muscular dystrophy, or limb-girdle muscular dystrophy (LGMD) such as LGMD type 1 or LGMD type 2), myasthenia gravis, myopathy (e.g., X-linked myotubular myopathy), myositis, peripheral neuropathy, or spinal muscular atrophy. Muscle diseases and disorders can be characterized and identified, e.g., through laboratory tests and/or evaluation by a clinician. In some embodiments, the subject has or is suspected of having a disease involving muscle cells (e.g., a disease caused by a defect, such as a genetic mutation, in one or more muscle cells or genes associated therewith). In some embodiments, a nucleic acid isolated or derived from the subject (e.g., genomic DNA, mRNA, or cDNA from the subject) is identified via sequencing (e.g., Sanger or next-generation sequencing) to comprise a mutation (e.g., in a gene associated with muscle development, health, maintenance, or function).

In some embodiments, a gene associated with muscle development, health, maintenance, or function is dystrophin/DMD, SCN4A, DMPK, ACTA, TPM3, TPM2, TNNT1, CFL2, KBTBD13, KLHL30, KKLHL3, KLHL41, LMOD3, MYPN, MTM1, nebulin, DNM2, TTN, RYR1, MYH7, TK2, GAA (α-glucosidase), CIC1, LMNA, CAV3, DNAJB6, TRIM32, desmin, LAMA2, COL6A1, COL6A2, COL6A3, or DUX4. In some embodiments the gene is dystrophin (DMD) or MTM1. In some embodiments, the gene is a gene in which mutations have been shown to cause limb-girdle muscular dystrophy (e.g., LGMD1 or LGMD2), such as MYOT, LMNA, CAV3, DNAJB6, DES, TNP03, HNRNPDL, CAPN3, DYSF, SGCG, SGCA, SGCB, SGCD, TCAP, TRIM32, FKRP, TTN, POMT1, ANO5, FKTN, POMT2, POMGnT1, DAG1, PLEC1, DES, TRAPPC11, GMPPB, ISPD, GAA, LIMS2, BVES, or TOR1A1P1. In some embodiments, a subject comprises a mutant form of one or more genes associated with muscle development, health, maintenance or function. In some embodiments, methods disclosed herein provide a cell (e.g., a muscle cell) of a subject with a functional form of a gene associated with muscle development, health, maintenance, or function.

### EXAMPLES

The following examples are included to demonstrate illustrative embodiments of the invention and are not considered limiting. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

### Example 1. Development of Capsid-Modified Next Generation AAVrh74 Vectors with Increased Transduction Efficiency in Primary Human Skeletal Muscle Cells: Implications in Gene Therapy of Muscular Dystrophies

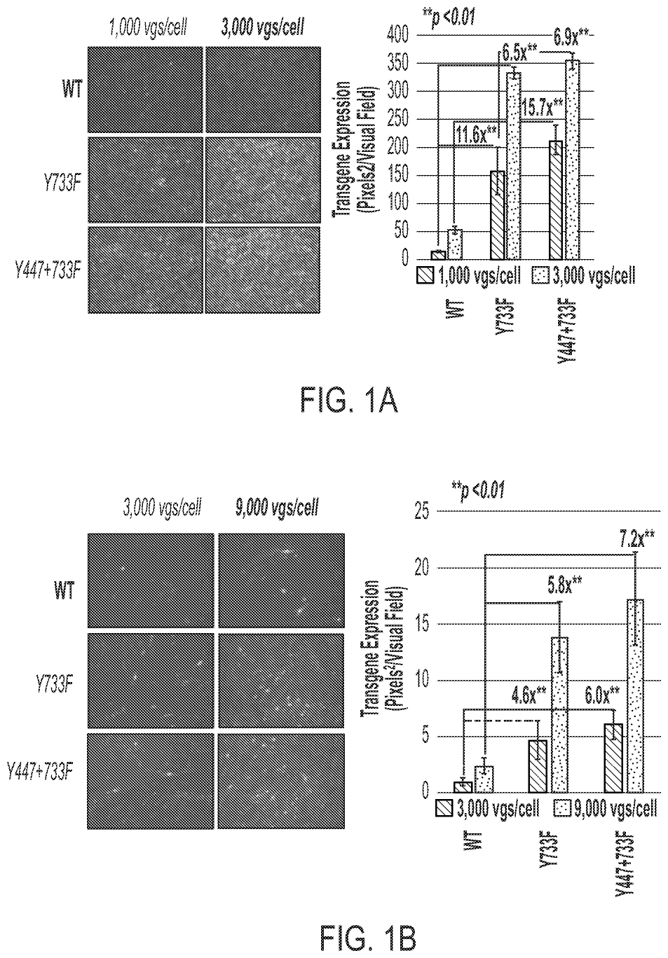

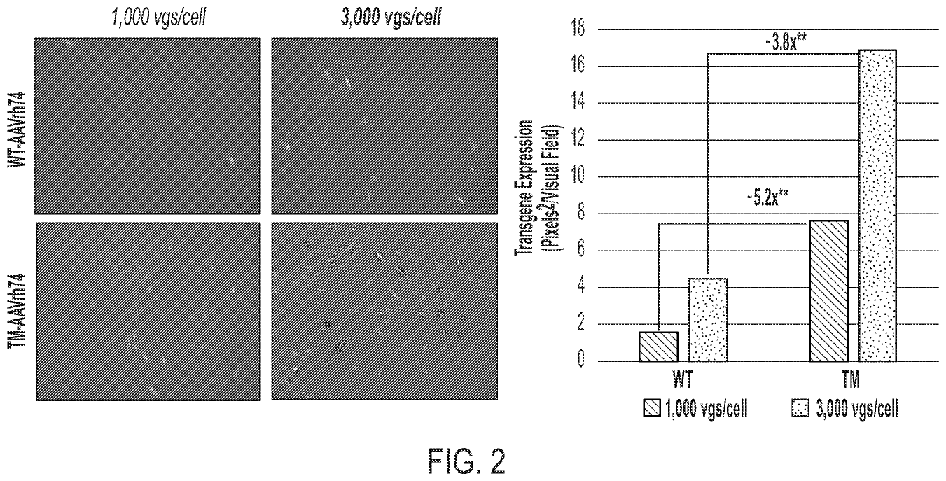

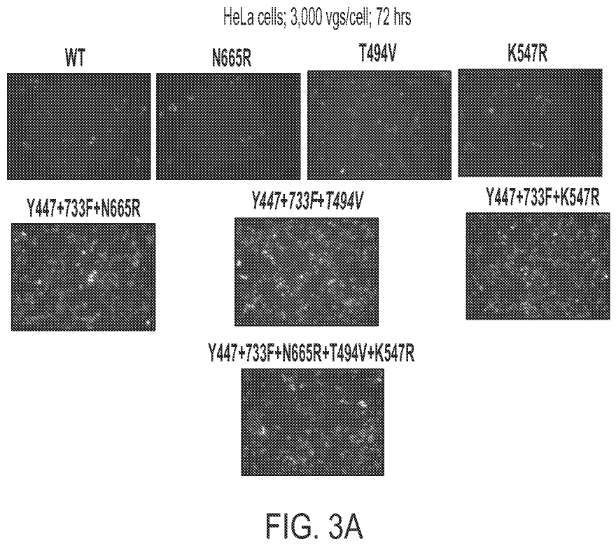

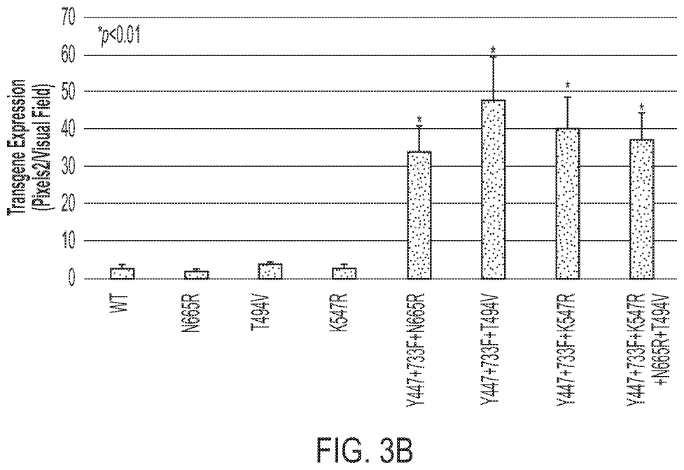

It has become increasingly clear that the host immune response to AAVs correlates directly with the AAV vector dose administered. For example, whereas a dose of up to 1×10¹⁴ vgs/kg of AAV8 vectors has been shown to be safe, a dose of 3×10¹⁴ vgs/kg has been associated with severe complications in 3 patients, complications which proved fatal for two of the patients, in a gene therapy trial of X-linked myotubular myopathy (Hum. Gene Ther., 31: 787, 2020). Although a dose of 2×10¹⁴ vgs/kg of AAVrh74 vectors has been shown to be well-tolerated in patients with Duchenne muscular dystrophy (JAMA Neurol., 77: 1122-1131, 2020), it would be desirable to achieve clinical efficacy at a significantly lower vector dose. It was previously reported that site-directed mutagenesis of specific surface-exposed tyrosine (Y) residues to phenylalanine (F) results in next generation (“NextGen”) AAV2 vectors that are significantly more efficient at reduced doses (Proc. Natl. Acad. Sci. USA, 105: 7827-7832, 2008; Mol. Ther., 18: 2048-2056, 2010), and are less immunogenic (Blood, 8: 121: 2224-2233, 2013). Because most, if not all, surface-exposed Y residues are conserved in AAVrh74, corresponding Y733F single-mutant (“SM”) and Y733+447F double-mutant (DM) AAVrh74 vectors were generated. The transduction efficiency of these vectors expressing an EGFP reporter gene was up to ˜12-fold and ˜16-fold higher, respectively, than that of the conventional wild-type (“WT”) AAVrh74 vectors in Hela cells (FIG. 1A). The Y-F mutant vectors were also significantly more efficient in transducing immortalized mouse myoblast cells of the C2C12 cell line (FIG. 1B). It was previously reported that inclusion of site-directed mutagenesis of surface-exposed threonine (T) to valine (V) residues further augments the transduction efficiency of AAV2 vectors (PLOS One, 8: e59142, 2013), so a Y733+Y447F+T494V triple-mutant (“TM”) ssAAVrh74 vector was additionally generated, which was up to ˜5-fold more efficient than the first generation ssAAVrh74 vector in primary human skeletal muscle cells (FIG. 2). Furthermore, single mutant T494V, K547R, and N665R. triple mutant Y447+733F+N665R and Y447+733F+K547R, and quintuple mutant Y447+733F+N665R+T494V+K547R ssAAVrh74 vectors were generated and tested for their transduction efficiencies. Each of the triple mutants showed increased transduction efficiency of Hela cells relative to the wild-type ssAAVrh74 vector, as did the quintuple mutant, and the transduction efficiencies of each of these multiple mutants were similar to the Y733+447F+T494V triple mutant (FIGS. 3A and 3B). Studies are currently underway to evaluate the efficacy of the mutant ssAAVrh74 vectors in skeletal muscle in a murine model in vivo. Taken together, these studies suggest that the use of NextGen AAVrh74 vectors may lead to the potentially safe and effective gene therapy of human muscular dystrophies at reduced doses, without the need for immune-suppression.

### Example 2. Development of Genome-Modified Generation x Single-Stranded AAVrh74 Vectors with Improved Transgene Expression in Primary Human Skeletal Muscle Cells

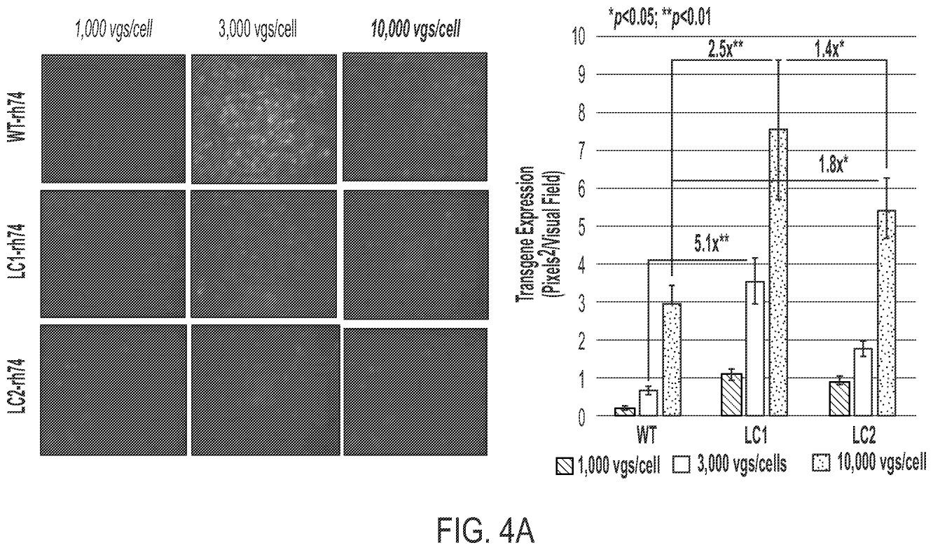

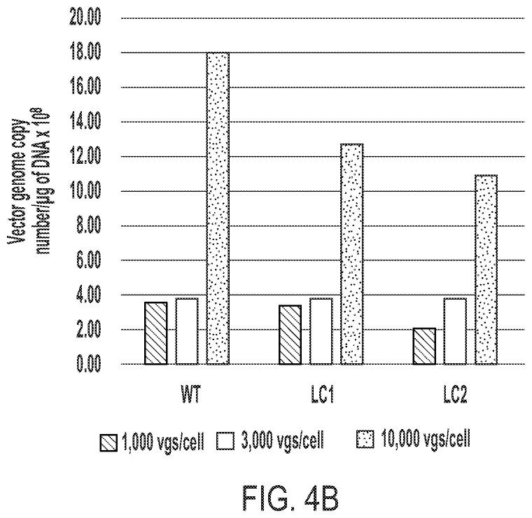

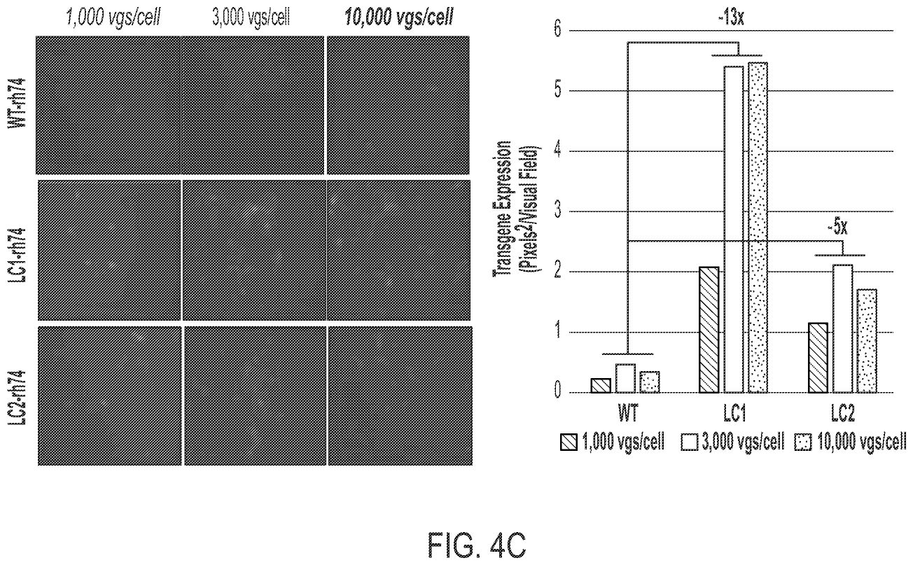

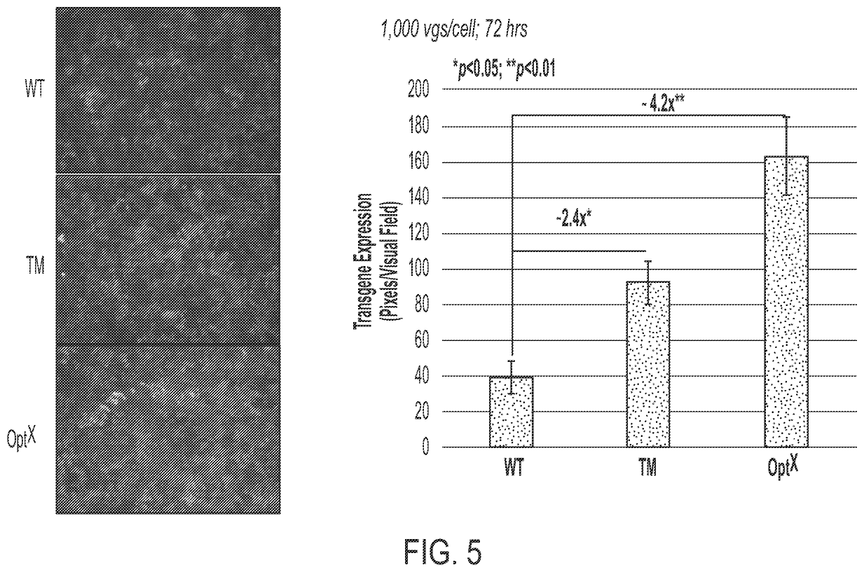

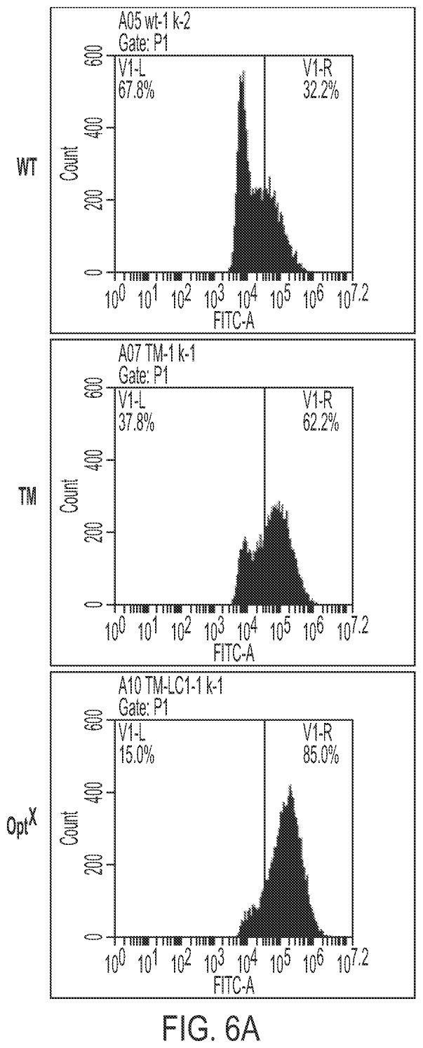

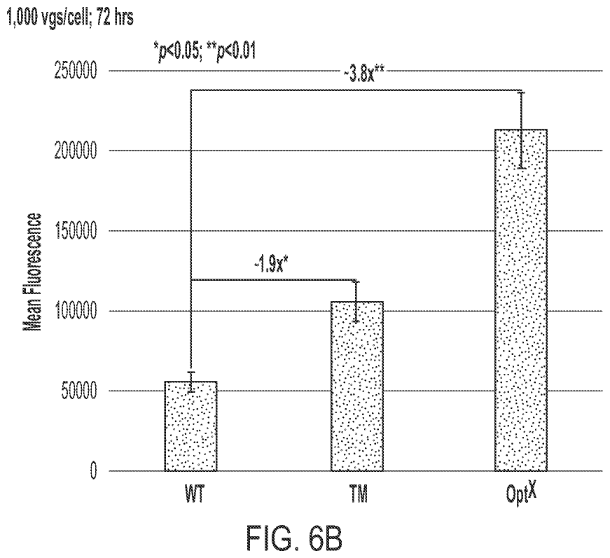

The naturally occurring AAV contains a single-stranded DNA genome, and expresses the viral genes poorly, because ssDNA is transcriptionally inactive, and there is no RNA polymerase that can transcribe a ssDNA. Similarly, transgene expression levels from recombinant ssAAV vectors are also negatively impacted. It was previously reported that the D-sequence in the AAV inverted terminal repeat (ITR) at the 3′-end of the vector genome plays a significant role in limiting transgene expression from ssAAV vectors (Proc. Natl. Acad. Sci. USA, 94: 10879-10884, 1997). A binding site was identified for the NF-κB negative regulatory factor (NRF), known to suppress transcription, in the D-sequence in the AAV-ITR. Substitution of the D-sequence with an S-sequence in the left ITR (LC1), or the right ITR (LC2) resulted in generation X (“GenX”) ssAAV vectors, which mediated up to 8-fold improved transgene expression (J. Virol., 89: 952-961, 2015). In the present study, it was evaluated whether encapsidation of these modified ssAAV genomes in AAVrh74 capsids would also lead to increased transgene expression. HeLa cells were transduced with WT, LC1, and LC2 vectors expressing the hrGFP reporter gene at multiplicities of infection of 1,000, 3,000, and 10,000 vgs/cell, and hrGFP fluorescence was quantitated 72 hours post-transduction. These results, shown in FIG. 4A, document ˜5 and ˜2.5-fold increase in transgene expression mediated by LC1 and LC2 vectors, respectively (p<0.01) relative to ssAAVrh74 vectors encapsidating genomes without D-sequence substitutions. The observed increase in transgene expression was not due to increased entry of LC1 and LC2 vectors, as documented by approximately similar numbers of the vector genomes quantitated by qPCR analyses of low molecular weight DNA samples isolated from transduced cells with each of these vectors (FIG. 4B). The extent of the transgene expression from these vectors was also evaluated in primary human skeletal muscle cells transduced at multiplicities of infection of 1,000, 3,000, and 10,000 vgs/cell of each of these vectors. Quantitation of fluorescence images indicated that ssLC1-AAVrh74 vectors averaged ˜13-fold increase, and ssLC2-AAVrh74 vectors averaged ˜5-fold increase in transgene expression compared with that from the conventional ssAAVrh74 vectors (FIG. 4C). Based on previously published studies with NextGen AAV2 and AAV3 serotype vectors (Hum. Gene Ther. Meth., 27: 143-149, 2016), it was anticipated that encapsidation of LC1 and LC2 GenX AAV genomes into NextGen AAVrh74 capsids would be feasible to achieve significantly higher levels of transgene expression in a murine model in vivo. To test the efficacy of such vectors, Y733+Y447F+T494V triple-mutant (“TM”) ssAAVrh74 vector comprising a substitution of the D-sequence of the left ITR with an S-sequence were generated and compared with TM ssAAVrh74 without genome modification and WT ssAAVrh74 vectors. The results in FIG. 5 and FIGS. 6A-6B show that the TM/D-sequence combined mutant ssAAVrh74 vector (“Opt^{X}”) showed ˜4-fold higher transgene expression in Hela cells relative to WT ssAAVrh74 vector, and ˜2-fold higher transgene expression than the TM ssAAVrh74 (without D-sequence substitutions), as measured by fluorescence microscopy imaging (FIG. 5) and flow cytometry (FIGS. 6A-6B) of hrGFP expressed from the vectors. These observations have significant implications in the potential use of GenX AAVrh74 vectors at further reduced doses in gene therapy of muscular dystrophies.

### Example 3. Development of Optimized (Opt^{X}) AAVrh74 Vectors with Increased Transduction Efficiency in Primary Human Skeletal Muscle Cells in Vitro and in Mouse Muscles in Vivo Following Systemic Administration

In one phase I/II clinical trial using AAV9 vectors, serious adverse events such as complement activation and thrombocytopenia causing renal damage and cardiopulmonary insufficiency were reported. In another trial, also using AAV9 vectors, several serious adverse events such as acute kidney injury involving atypical hemolytic uremic syndrome and thrombocytopenia, and more recently, the death of a patient, were also reported. Sarepta Therapeutics reported the results of a phase I/II trial using AAVrh74 vectors with vomiting as the only adverse event, indicating that AAVrh74 vectors are safer, even at the high dose of 2×10¹⁴ vgs/kg used.

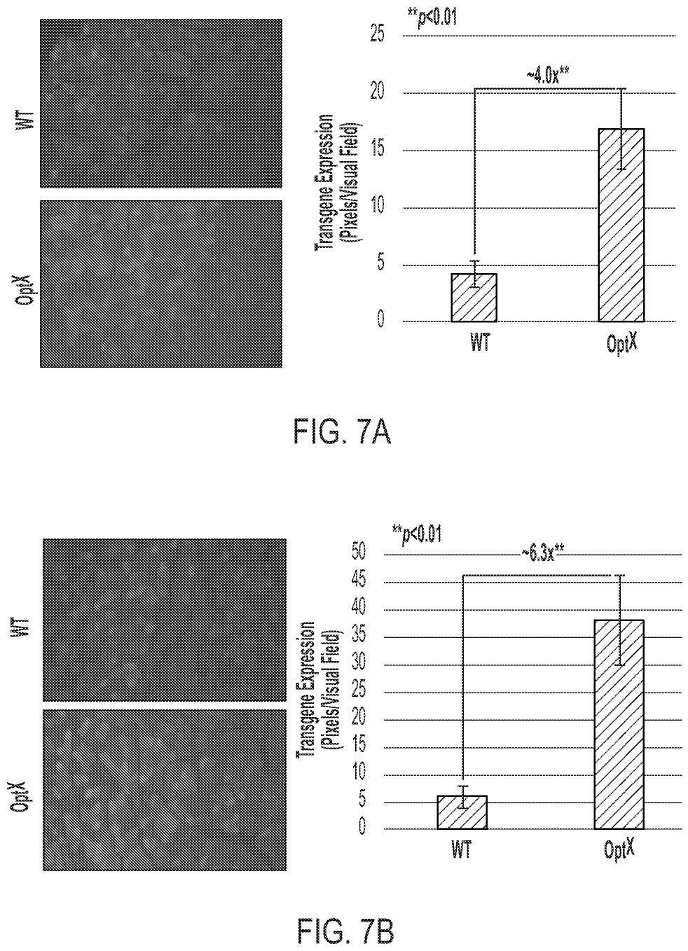

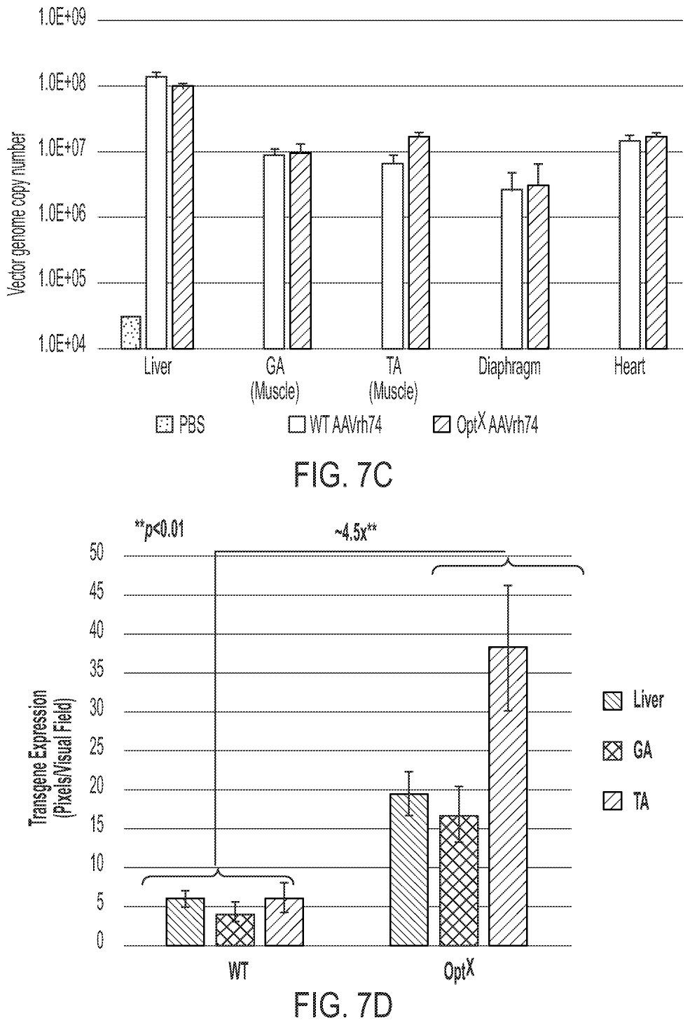

As described in the preceding Examples, capsid-modified next generation (“NextGen”) AAVrh74 vectors and genome-modified generation X (“GenX”) AAVrh74 vectors are significantly more efficient than their wild-type (WT) counterpart (see also Mol. Ther., 29: 159-160, 2021; Mol. Ther., 29: 184-185, 2021). In the present Example, the two modifications were combined to generate optimized (“Opt^{X}”) AAVrh74 vectors. The transduction efficiency of Opt^{X} AAVrh74 vectors was evaluated in primary human skeletal muscle cells in vitro. Results demonstrated that transduction efficiency of these cells was up to about 5-fold higher than that of wild-type AAVrh74 vectors. The efficacy of the WT and the Opt^{X} AAVrh74 vectors was also evaluated in mouse muscles in vivo following systemic administration. FIGS. 7A-7D demonstrate that the transduction efficiency of the Opt^{X} AAVrh74 vectors was about 5-fold higher in gastrocnemius (GA; FIG. 7A) and tibialis anterior (TA; FIG. 7B) muscles. Interestingly, the total genome copy numbers of either the WT or Opt^{X} AAVrh74 vectors in GA, TA, diaphragm and heart muscles were not significantly different from one another (FIG. 7C), suggesting that the observed increase in transduction efficiency of the Opt^{X} AAVrh74 vectors may have resulted from improved intracellular trafficking and nuclear transport of these vectors.

Taken together, these studies suggest that the use of Opt^{X} AAVrh74 vectors may lead to safe and effective gene therapy of human muscular dystrophies at reduced doses.

### Example 4. Development of Genome-Modified Generation Y (GenY) AAVrh74 Vectors with Improved Transgene Expression in a Mouse Skeletal Muscle Cell Line and in Primary Human Skeletal Muscle Cells

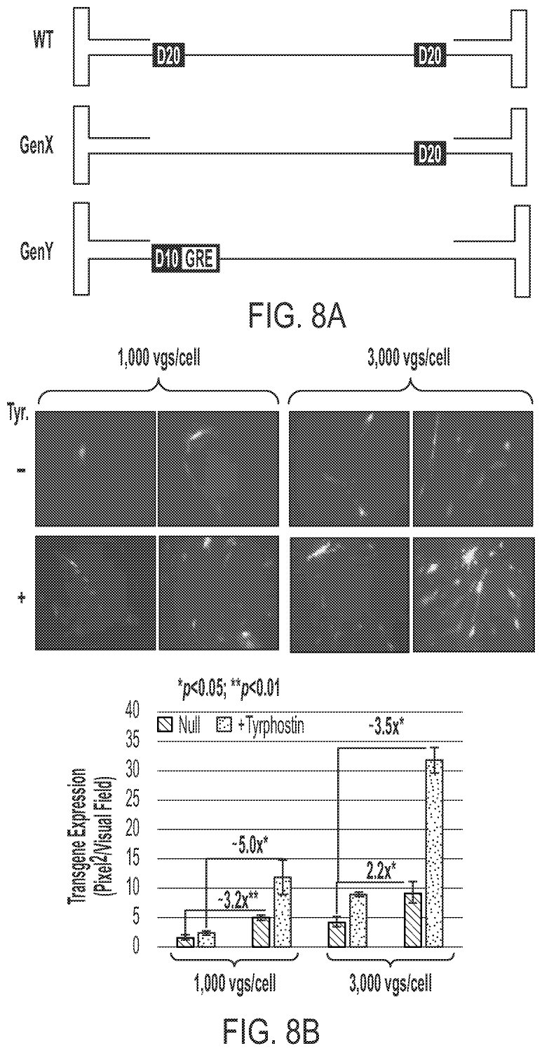

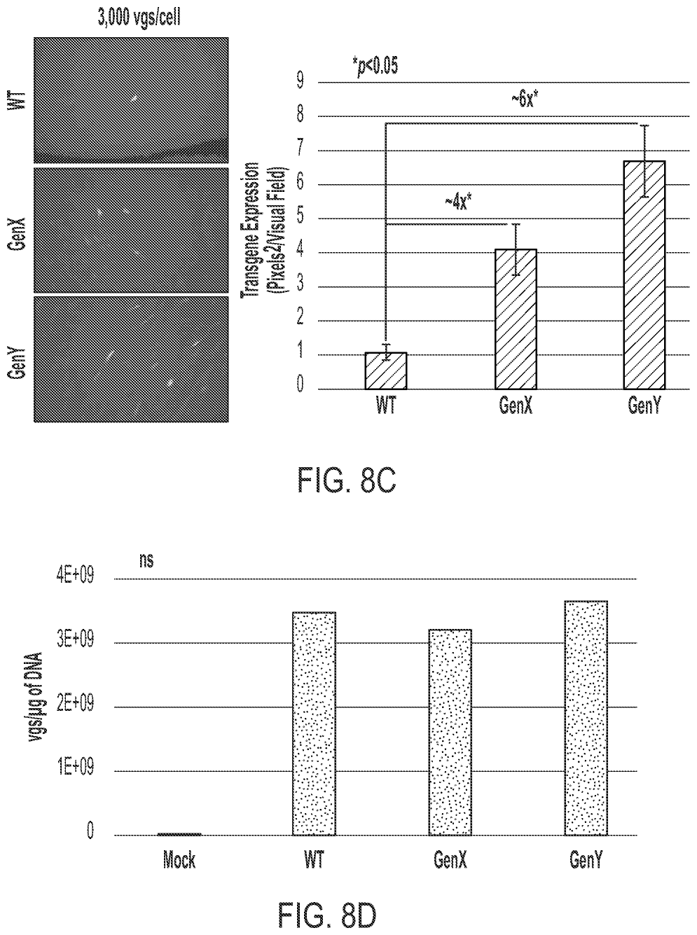

Transgene expression levels from recombinant ssAAV vectors are typically relatively low as a result of ssDNA being transcriptionally inactive. Substitution of the D-sequence in the left inverted terminal repeat (ITR) of AAV vectors to form “Generation X” (“GenX”) AAV vectors results in AAV vectors which mediate up to 8-fold improved transgene expression (J. Virol., 89: 952-961, 2015). The extent of transgene expression from GenX AAVrh74 vectors is also ˜5-fold higher than that from wild-type (WT) AAVrh74 vectors (Mol. Ther., 29: 184-185, 2021). The distal 10 nucleotides in the AAV2 D-sequence share partial homology to the consensus half-site of the glucocorticoid receptor-binding element (GRE), and the glucocorticoid receptor signaling pathway is activated following AAV2 infection or AAV2 vector transduction (Mol. Ther., 24: S6, 2016). In the current Example, substitution of the distal (with respect to the terminus of the nucleic acid vector) 10 nucleotides in the D-sequence with the authentic GRE was evaluated for its ability to increase transgene expression from AAVrh74 vectors, termed “Generation Y” (“GenY”) vectors, shown schematically in FIG. 8A. Transgene expression from the WT and GenY AAVrh74 vectors was evaluated in C2C12 mouse skeletal muscle cells. GenY AAVrh74 vectors averaged about 2-3-fold increase in transgene expression compared with WT AAVrh74 vectors (FIG. 8B). Transgene expression was further increased by about 4-5-fold following pre-treatment with tyrphostin, a specific inhibitor of cellular epidermal growth factor receptor protein tyrosine kinase (FIG. 8B). WT, GenX, and GenY vectors were also evaluated in primary human skeletal muscle cells. Transgene expression from the GenX and the GenY AAVrh74 vectors was about 4-fold and about 6-fold higher, respectively, compared with WT AAVrh74 vectors (FIG. 8C). Analysis by qPCR of low molecular weight DNA samples isolated from primary human skeletal muscle cells transduced with WT, GenX, or GenY AAVrh74 vectors showed similar vector genome copy numbers in cells transduced with each vector (FIG. 8D), indicating that the observed increase in transgene expression did not result from increased entry of the GenX or the GenY vectors.

These studies suggest that the combined use of the capsid-modified NextGen+GenY (Opt^{Y}) AAVrh74 vectors may further reduce the need for the use of high vector doses, which has significant implications in the potential use of Opt^{Y} AAVrh74 vectors in the safe and effective gene therapy of muscular dystrophies in humans.

### Example 5. In Vivo Efficacy of Opt^{X} and Opt^{Y} AAVrh74 Vectors

In this Example, the efficacy of of AAVrh74 vectors comprising Y733+Y447F+T494V triple-mutant (TM) capsids and either a GenX (with substitution of the D-sequence with an S-sequence in the left ITR) or a GenY (with substitution of a GRE sequence in the left ITR replacing a portion of the D-sequence) modified genome was tested. The TM+GenX vector is referred to as “Opt^{X}” and the TM+GenY vector is referred to as “Opt^{Y}”.

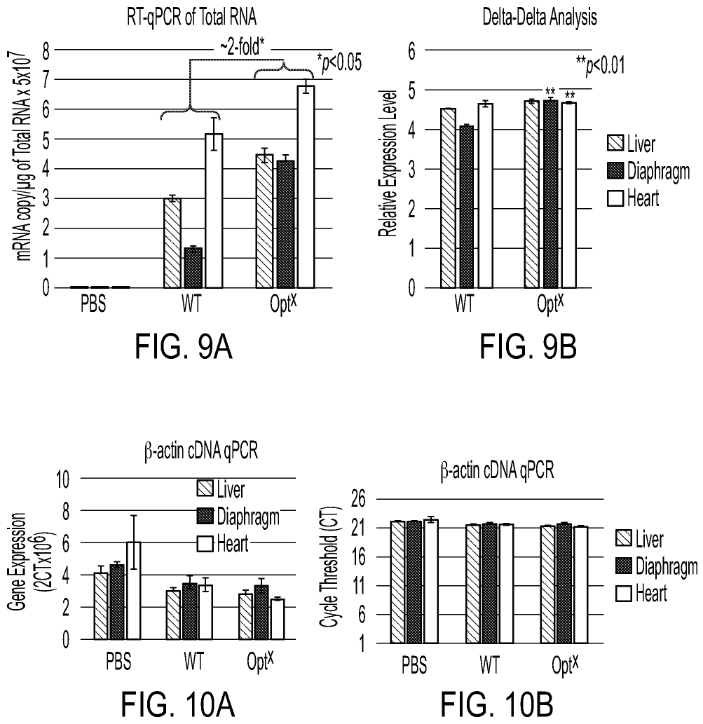

To test the Opt^{X} vector, C57BL/6 mice were administered intravenously either PBS, a dose of wild-type AAVrh74 particles (“WT”) or a dose of Opt^{X} AAVrh74 particles (“Opt^{X}”). The doses of WT and Opt^{X} particles were equivalent to 1×10¹² viral genomes. Eight weeks following administration of the particles, various tissues were collected and RNA was extracted. Reverse transcription-quantitative PCR (RT-qPCR) was conducted for hrGFP mRNA expressed from the vectors. FIG. 9A shows the amount of hrGFP mRNA per μg total RNA in liver (diagonally striped bars), diaphragm (solid bars), and heart (open bars). The results demonstrate that the Opt^{X} AAVrh74 vector achieved approximately 2-fold higher hrGFP expression in the mouse tissues relative to WT AAVrh74, when values are aggregated across the tested tissues. FIG. 9B shows that the transgene expression from Opt^{X} AAVrh74 vectors in the diaphragm and the heart, but not the liver, was significantly higher than the transgene expression from WT AAVrh74 vectors when calculated relative to endogenous β-actin gene expression.

FIGS. 10A and 10B show expression of β-actin mRNA in the samples from mice administered PBS, WT AAVrh74 particles, or Opt^{X} AAVrh74 particles. The results demonstrate no difference in β-actin expression between the various samples, showing that the increased hrGFP expression measured in samples from Opt^{X} particle-treated mice are due to improved properties of the particles.

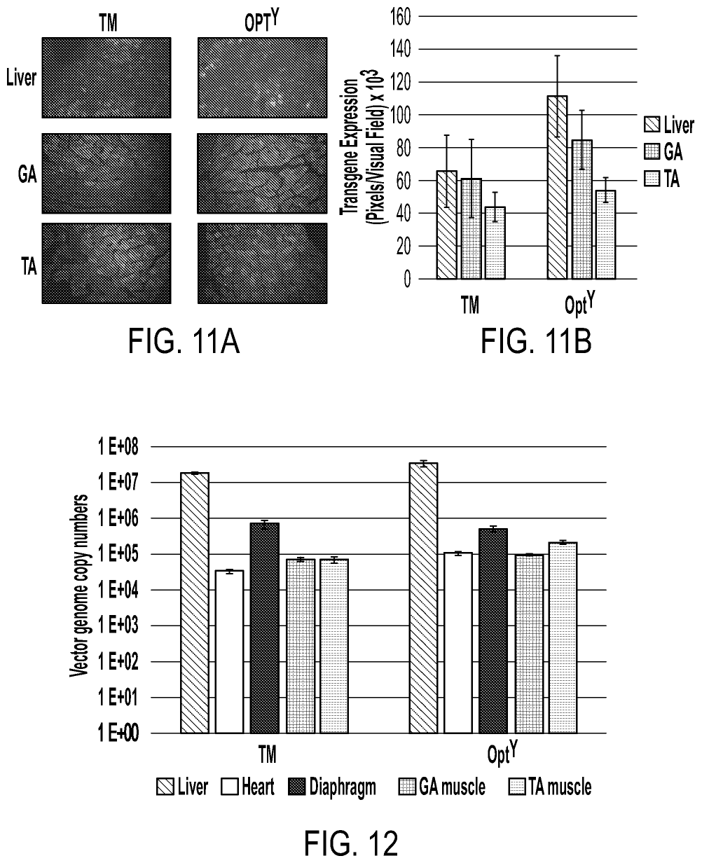

To test the Opt^{Y} vector, C57BL/6 mice were administered intravenously either PBS, a dose of AAVrh74 particles with TM capsid proteins (“TM”) or a dose of Opt^{Y} AAVrh74 particles (“Opt^{Y}”). The doses of AAVrh74 particles were equivalent to 1×10¹² viral genomes. Eight weeks following administration of the particles, various tissues were collected. Tissue sections were prepared and RNA was extracted. Fluorescence microscopy of tissue sections demonstrated increased hrGFP fluorescence in liver, gastrocnemius (“GA”) and tibialis anterior (“TA”) after administration of Opt^{Y} particles relative to TM-only particles (FIG. 11A; fluorescence quantified in FIG. 11B).

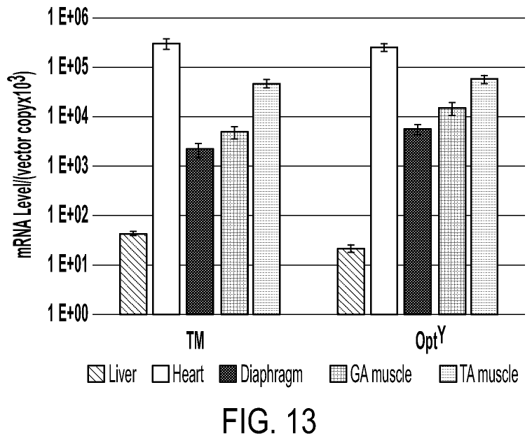

The results shown in FIG. 12 demonstrate that the copy number of vector genomes in liver (diagonally striped bars), heart (open bars), diaphragm (filled bars), gastrocnemius (“GA muscle”; square patterned bars), and tibialis anterior (“TA muscle”; horizontal striped bars) was not significantly different in mice treated with TM-only AAVrh74 particles (“TM”) versus mice treated with Opt^{Y} particles (“Opt^{Y}”). By contrast, hrGFP mRNA expression from the AAVrh74 vectors was different in certain tissues. As shown in FIG. 13, hrGFP expression was decreased in liver, increased in diaphragm, increased in gastrocnemius, and slightly increased in tibialis anterior in Opt^{Y} particle-treated mice relative to TM-only particle-treated mice.

The results presented in this Example demonstrate that Opt^{X} and Opt^{Y} AAVrh74 vectors are capable of achieving improved transgene expression profiles in vivo after intravenous administration to mice.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of,” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A capsid protein comprising an amino acid substitution at a position corresponding to Y447, T494, K547, N665, and/or Y733 of the wild-type AAVrh74 capsid protein of SEQ ID NO: 1, wherein the capsid protein is an AAVrh74 serotype capsid protein,
optionally wherein the substitution is Y447F, T494V, K547R, N665R, and/or Y733F.

2. An AAVrh74 particle comprising the capsid protein of claim 1.

3. The AAVrh74 particle of claim 2, further comprising a nucleic acid vector, wherein the nucleic acid vector comprises a first inverted terminal repeat (ITR) comprising a first D-sequence and a second ITR comprising a second D-sequence, wherein the first D-sequence or the second D-sequence is substituted with an S-sequence,
optionally wherein the S-sequence comprises, consists essentially of, or consists of the nucleotide sequence TATTAGATCTGATGGCCGCT (SEQ ID NO: 17).

4. The AAVrh74 particle of claim 2, further comprising a nucleic acid vector, wherein the nucleic acid vector comprises a first inverted terminal repeat (ITR) comprising a first D-sequence and a second ITR comprising a second D-sequence, wherein the first D-sequence and/or the second D-sequence is substituted with a glucocorticoid receptor-binding element (GRE),
optionally wherein the GRE comprises, consists essentially of, or consists of the nucleotide sequence AGAACANNNTGTTCT (SEQ ID NO: 18), or its reverse or reverse complement, wherein each N is independently a T, C, G, or A.

5. A composition comprising the capsid protein of claim 1.

6. A composition comprising the AAVrh74 particle of any one of claims 2-4

7. A method comprising contacting a cell with a composition comprising an AAVrh74 particle, wherein the AAVrh74 particle comprises a capsid protein and a nucleic acid vector,
(i) wherein the capsid protein comprises an amino acid substitution at a position corresponding to Y447, T494, K547, N665, and/or Y733 of the wild-type AAVrh74 capsid protein of SEQ ID NO: 1, optionally wherein the substitution is Y447F, T494V, K547R, N665R, and/or Y733F, and/or
(ii) wherein the nucleic acid vector comprises a first inverted terminal repeat (ITR) comprising a first D-sequence and a second ITR comprising a second D-sequence, wherein the first D-sequence and/or the second D-sequence is substituted with an S-sequence, optionally wherein the S-sequence comprises, consists essentially of, or consists of the nucleotide sequence TATTAGATCTGATGGCCGCT (SEQ ID NO: 17)

8. A method comprising contacting a cell with a composition comprising an AAVrh74 particle, wherein the AAVrh74 particle comprises a capsid protein and a nucleic acid vector,
(i) wherein the capsid protein comprises an amino acid substitution at a position corresponding to Y447, T494, K547, N665, and/or Y733 of the wild-type AAVrh74 capsid protein of SEQ ID NO: 1, optionally wherein the substitution is Y447F, T494V, K547R, N665R, and/or Y733F, and/or
(ii) wherein the nucleic acid vector comprises a first inverted terminal repeat (ITR) comprising a first D-sequence and a second ITR comprising a second D-sequence, wherein the first D-sequence and/or the second D-sequence is substituted with a glucocorticoid receptor-binding element (GRE), optionally wherein the GRE comprises, consists essentially of, or consists of the nucleotide sequence AGAACANNNTGTTCT (SEQ ID NO: 18), or its reverse or reverse complement, wherein each N is independently a T, C, G, or A.

9. The method of claim 7 or 8, wherein the capsid protein comprises an amino acid substitution at a position corresponding to Y447, T494, K547, N665, and/or Y733 of the wild-type AAVrh74 capsid protein of SEQ ID NO: 1, optionally wherein the substitution is Y447F, T494V, K547R, N665R, and/or Y733F.

10. The method of claim 7, wherein the nucleic acid vector comprises the first ITR and the second ITR, wherein the first D-sequence or the second D-sequence is substituted with the S-sequence, optionally wherein the S-sequence comprises, consists essentially of, or consists of the nucleotide sequence TATTAGATCTGATGGCCGCT (SEQ ID NO: 17).

11. The method of claim 8, wherein the nucleic acid vector comprises the first ITR and the second ITR, wherein the first D-sequence and/or the second D-sequence is substituted with the GRE, optionally wherein the GRE comprises, consists essentially of, or consists of the nucleotide sequence AGAACANNNTGTTCT (SEQ ID NO: 18), or its reverse or reverse complement, wherein each N is independently a T, C, G, or A.

12. The method of claim 7, wherein the capsid protein comprises an amino acid substitution at a position corresponding to Y447, T494, K547, N665, and/or Y733 of the wild- type AAVrh74 capsid protein of SEQ ID NO: 1, and
wherein the nucleic acid vector comprises the first ITR and the second ITR, wherein the first D-sequence or the second D-sequence is substituted with the S-sequence,
optionally wherein the substitution is Y447F, T494V, K547R, N665R, and/or Y733F, and
optionally wherein the S-sequence comprises, consists essentially of, or consists of the nucleotide sequence TATTAGATCTGATGGCCGCT (SEQ ID NO: 17).

13. The method of claim 8, wherein the capsid protein comprises an amino acid substitution at a position corresponding to Y447, T494, K547, N665, and/or Y733 of the wild-type AAVrh74 capsid protein of SEQ ID NO: 1, and
wherein the nucleic acid vector comprises the first ITR and the second ITR, wherein the first D-sequence and/or the second D-sequence is substituted with the GRE,
optionally wherein the substitution is Y447F, T494V, K547R, N665R, and/or Y733F, and
optionally wherein the GRE comprises, consists essentially of, or consists of the nucleotide sequence AGAACANNNTGTTCT (SEQ ID NO: 18), or its reverse or reverse complement, wherein each N is independently a T, C, G, or A.

14. The method of any one of claims 7-13, wherein the capsid protein comprises amino acid substitutions at positions corresponding to:
(a) Y447 and Y733, optionally wherein the substitutions are Y447F and Y733F;
(b) Y447, Y733, and N665, optionally wherein the substitutions are Y447F, Y733F, and N665R;
(c) Y447, Y733, and T494, optionally wherein the substitutions are Y447F, Y733F, and T494V;
(d) Y447, Y733, and K547, optionally wherein the substitutions are Y447F, Y733F, and K547R; or
(e) Y447, Y733, N665, T494, and K547, optionally wherein the substitutions are Y447F, Y733F, N665R, T494V, and K547R,
of the wild-type AAVrh74 capsid protein of SEQ ID NO: 1.

15. The method of any one of claims 7-13, wherein the first ITR and the second ITR are each an AAV2 serotype ITR or an AAV3 serotype ITR.

16. The method of any one of claims 7-15, wherein the first D-sequence is substituted with the S-sequence, or wherein the first D-sequence is substituted with the GRE.

17. The method of any one of claims 7-15, wherein the second D-sequence is substituted with the S-sequence, or wherein the second D-sequence is substituted with the GRE.

18. The method of any one of claims 7-17, wherein the S-sequence comprises, consists essentially of, or consists of the nucleotide sequence TATTAGATCTGATGGCCGCT (SEQ ID NO: 17), or
wherein the GRE comprises, consists essentially of, or consists of the nucleotide sequence AGAACANNNTGTTCT (SEQ ID NO: 18), or its reverse or reverse complement, wherein each N is independently a T, C, G, or A.

19. The method of any one of claims 7-18, wherein the transduction efficiency of the AAVrh74 particle is at least two-fold higher than a wild-type AAVrh74 particle.

20. The method of any one of claims 7-19, wherein the packaging efficiency of the AAVrh74 particle is decreased relative to a wild-type AAVrh74 particle.

21. The method of any one of claims 7-20, wherein the composition further comprises a pharmaceutically-acceptable carrier.

22. The method of any one of claims 7-21, wherein the cell is a mammalian cell.

23. The method of any one of claims 7-22, wherein the cell is a muscle cell.

24. The method of any one of claims 7-23, wherein the cell is a skeletal muscle cell.

25. The method of any one of claims 7-23, wherein the cell is a gastrocnemius cell or a tibialis anterior cell.

26. The method of any one of claims 7-25, wherein the nucleic acid vector comprises a regulatory element.

27. The method of claim 26, wherein the regulatory element comprises a promoter, an enhancer, a silencer, an insulator, a response element, an initiation site, a termination signal, or a ribosome binding site.

28. The method of claim 27, wherein the promoter is a constitutive promoter.

29. The method of claim 27, wherein the promoter is an inducible promoter.

30. The method of any one of claims 27-29, wherein the promoter is a tissue-specific promotor, a cell type-specific promoter, or a synthetic promoter.

31. The method of any one of claims 7-30, wherein the nucleic vector comprises a nucleotide sequence of a gene of interest.

32. The method of claim 31, wherein the gene of interest encodes a therapeutic protein or a diagnostic protein.

33. The method of any one of claims 7-32, wherein the contacting is in vivo.

34. The method of claim 33, further comprising administering the composition comprising the AAVrh74 particle to a subject.

35. The method of claim 34, wherein the cell is in the subject.

36. The method of claim 34 or 35, wherein the subject is human.

37. The method of claim 34, 35, or 36, wherein the subject is at risk of or suffering from a muscle disease, optionally wherein the muscle disease is amyotrophic lateral sclerosis, Charcot-Marie-Tooth disease, multiple sclerosis, muscular dystrophy, myasthenia gravis, myopathy, myositis, peripheral neuropathy, or spinal muscular atrophy.

38. The method of claim 37, wherein the muscle disease is Duchenne muscular dystrophy, optionally wherein the subject has a mutation in a dystrophin gene.

39. The method of claim 37, wherein the muscle disease is limb-girdle muscular dystrophy.

40. The method of claim 37, wherein the muscle disease is X-linked myotubular myopathy, optionally wherein the subject has a mutation in a MTM1 gene.

41. The method of any one of claims 34-37, wherein the composition is administered to the subject by subcutaneous injection, by intramuscular injection, by intravenous injection, by intraperitoneal injection, or orally.

42. The method of any one of claims 7-32, wherein the contacting is in vitro or ex vivo.


SEQUENCE LISTING (36 sequences)

SEQ ID NO: 1 - protein, 738 aa, Artificial Sequence
  Synthetic
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - protein, 736 aa, Unknown
  AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - protein, 735 aa, Unknown [sequence omitted]

SEQ ID NO: 4 - protein, 736 aa, Unknown
  AAV3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 5 - protein, 734 aa, Unknown
  AAV4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 6 - protein, 724 aa, Unknown
  AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 7 - protein, 736 aa, Unknown
  AAV6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 8 - protein, 737 aa, Unknown
  AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 9 - protein, 738 aa, Unknown
  AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 10 - protein, 736 aa, Unknown
  AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 738 aa, Unknown
  AAV10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 12 - DNA, 145 bp, Unknown
  AAV2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 13 - DNA, 146 bp, Unknown
  AAV3
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggc

SEQ ID NO: 14 - DNA, 167 bp, Unknown
  AAV5
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 15 - DNA, 142 bp, Unknown
  AAV6
ttgcccactccctctatgcgcgctcgctcgctcggtggggcctgcggaccaaaggtccgcagacggcagagctctgctctgccggccccaccgagcgagcgagcgcgcatagagggagtgggcaactccatcactaggggta

SEQ ID NO: 16 - DNA, 20 bp, Artificial Sequence
  Synthetic
ctccatcactaggggttcct

SEQ ID NO: 17 - DNA, 20 bp, Artificial Sequence
  Synthetic
tattagatctgatggccgct

SEQ ID NO: 18 - DNA, 15 bp, Artificial Sequence
  Synthetic
  misc_feature (7)..(9): n is a, c, g, or t
agaacannntgttct

SEQ ID NO: 19 - DNA, 15 bp, Artificial Sequence
  Synthetic
  misc_feature (7)..(9): n is a, c, g, or t
ggtacannntgtyct

SEQ ID NO: 20 - DNA, 15 bp, Artificial Sequence
  Synthetic
agaacaggatgttct

SEQ ID NO: 21 - DNA, 15 bp, Artificial Sequence
  Synthetic
ggcacagtgtggtct

SEQ ID NO: 22 - DNA, 2217 bp, Artificial Sequence
  Synthetic
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgccgatcctccgaccaccttcaatcaggccaagctggcttctttcatcacgcagtacagtaccggtcaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 23 - protein, 733 aa, Unknown
  AAV11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 24 - protein, 742 aa, Unknown
  AAV12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 25 - DNA, 2211 bp, Artificial Sequence
  Synthetic
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 26 - DNA, 2208 bp, Artificial Sequence
  Synthetic
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctccggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 27 - DNA, 2211 bp, Artificial Sequence
  Synthetic
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 28 - DNA, 2285 bp, Artificial Sequence
  Synthetic
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaataacctgttaatcaataaaccggtttattcgtttcagttgaactttggtctccgtgtccttcttatcttatctcgtttcc

SEQ ID NO: 29 - DNA, 2175 bp, Artificial Sequence
  Synthetic
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 30 - DNA, 2212 bp, Artificial Sequence
  Synthetic
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaat

SEQ ID NO: 31 - DNA, 2214 bp, Artificial Sequence
  Synthetic
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 32 - DNA, 2217 bp, Artificial Sequence
  Synthetic
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctggagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 33 - DNA, 2211 bp, Artificial Sequence
  Synthetic
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 34 - DNA, 2217 bp, Artificial Sequence
  Synthetic
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaa

SEQ ID NO: 35 - DNA, 143 bp, Unknown
  AAV1
ttgcccactccctctctgcgcgctcgctcgctcggtggggcctgcggaccaaaggtccgcagacggcagaggtctcctctgccggccccaccgagcgagcgagcgcgcagagagggagtgggcaactccatcactaggggtaa

SEQ ID NO: 36 - DNA, 146 bp, Unknown
  AAV4
ttggccactccctctatgcgcgctcgctcactcactcggccctggagaccaaaggtctccagactgccggcctctggccggcagggccgagtgagtgagcgagcgcgcatagagggagtggccaactccatcatctaggtttgccc